(19) United States
(12) Patent Application Publication
(10) Pub. No.: US 2026/0130701 A1
(43) Pub. Date: May 14, 2026

(54) CONNECTION MECHANISMS, SYSTEMS AND METHODS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Kevin W. Belew, Hernando, MS (US); Abel C. Kim, Cordova, TN (US); Michael D. Hollandsworth, Jr., Memphis, TN (US); Richard D. Lambert, Collierville, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/120,036

(22) PCT Filed: Nov. 8, 2023
(86) PCT No.: PCT/US2023/037025

§ 371 Date: Apr. 10, 2025

Related U.S. Application Data
(60) Provisional application No. 63/425,391, filed on Nov. 15, 2022.
(60) Provisional application No. 63/486,501, filed on Feb. 23, 2023.

(51) Int. Cl. A61B 17/92; A61B 17/00; A61F 2/46

(52) U.S. Cl. CPC A61B 17/921; A61F 2/4607; A61F 2/4609; A61B 2017/0046

(57) ABSTRACT

A connection mechanism for a tool and associated methods thereof. The connection mechanism includes a housing having a hollow interior, a spring-loaded movable collar portion positioned exterior to the hollow interior of the housing, and spring-loaded latches coupled to the housing. The spring-loaded latches include first retention mechanisms configured to releasably secure external objects within the hollow interior and prevent translational motion of the external objects once engaged by the spring-loaded latches. The collar portion includes a first portion of second retention mechanisms and the housing include a second portion of the second retention mechanisms. Upon interaction of the first and second portions of each of the second retention mechanisms, the second retention mechanisms is configured to releasably secure the external objects within the hollow interior and prevent rotational motion of the external objects once engaged by the collar.

### CROSS-REFERENCE TO RELATED APPLICATIONS

This is a non-provisional of, and claims the benefit of the filing date of, U.S. provisional patent application No. 63/425,391, filed Nov. 15, 2022, entitled “Quick Connect Collar Mechanism”, and U.S. provisional patent application No. 63/486,501, filed Feb. 23, 2023, entitled “Impact Drive Connection Adapter,” the entirety of each application is incorporated by reference herein.

### TECHNICAL FIELD

The present disclosure is generally directed to various connection mechanisms, systems and/or methods that may be used for coupling of tools, instruments, devices, etc., to an orthopedic surgical instrument or an impactor. In some examples, such connection mechanisms, systems and/or methods include quick connect mechanism(s), impact drive connection adaptor(s), and other connection devices.

### BACKGROUND

Orthopedic surgical procedures such as, for example, hip procedures, knee procedures, shoulder procedures, etc., have become common place in today's society. For example, total hip arthroplasty or hip replacement is a well-known procedure for repairing damaged bone (e.g., a damaged hip). During a total hip arthroplasty, an acetabular system may be implanted into a patient's acetabulum. In addition, and/or alternatively, a femoral implant may be implanted into a patient's femur. During the surgical procedure, the patient's bone typically needs to be prepared to receive the orthopedic implant. For example, a surgical tool such as, for example, an orthopedic broach, rasp, cutting tool, etc. (terms used interchangeably herein without the intent to limit or distinguish) may be used to prepare an inner surface of a patient's intramedullary canal to receive an orthopedic implant such as, for example, a femoral hip prosthesis, an intramedullary nail, etc. The preparation of the intramedullary canal by the surgeon is designed to insure a proper fit between the patient's femur and the implant. In addition, the orthopedic implant such as, for example, the acetabular cup, may need to be impacted into proper position. Moreover, during removal of the broach from the patient's intramedullary canal, the broach may become struck within the patient's intramedullary canal. To perform one or more of the above orthopedic surgical procedures, a powered instrument, such as, for example, a surgical impactor, may be used. The impactor can be coupled to one or more tools and can provide power to, for example, drive the tools forward, remove tools, etc.

During an orthopedic surgery, there is often a need to connect two instruments, tools, devices, etc. together via a connection mechanism, preferably a quick connect mechanism. Often this connection is overly complex in its operation due to strength and/or safety (e.g., accidental disengagement and/or other fail-safe) requirements. This can create frustration for the user, or potentially even a hazardous environment if the connection is unsafe due to its complexity. This problem is likely most severe in the field of orthopedic surgery but can also be seen in other applications of a quick connect mechanism, such as construction hand tools or power tools, weightlifting collars, and other industrial positioning or end stop collar applications.

Thus, it would be beneficial to provide connection mechanism that provides a stable and secure connection between various tools, devices, etc. It is with respect to these and other considerations that the present disclosure may be useful.

### SUMMARY

This summary is provided to introduce a selection of concepts in a simplified form that are further described below in the detailed description. This summary is not intended to identify key features or essential features of the claimed subject matter, nor is it intended as an aid in determining the scope of the claimed subject matter.

In some examples, the present disclosure relates to one or more connection mechanisms, apparatus, systems, and/or methods associated therewith. In one example, the connection mechanism(s) may include a quick connect collar mechanism for a tool, device, etc., such as, for example, an orthopedic impactor or impactor mechanism (terms used interchangeably herein without the intent to limit or distinguish). The quick connect collar mechanism may include a housing having a hollow interior, a spring-loaded movable collar portion positioned exterior to the hollow interior of the housing, and one or more spring-loaded latches coupled to the housing. The spring-loaded latches may include one or more first retention mechanisms configured to releasably secure one or more external objects within the hollow interior of the housing and prevent translational motion of the external objects once engaged by the spring-loaded latches. The collar portion may include a first portion of one or more second retention mechanisms and the housing may include a second portion of the one or more second retention mechanisms. Upon interaction of the first and second portions of each of the one or more second retention mechanisms, the second retention mechanisms may be configured to releasably secure the external objects within the hollow interior of the housing and prevent rotational motion of the external objects once engaged by the collar.

In any preceding or subsequent examples, the first portion of the second retention mechanisms may include one or more surfaces configured to interact with the second portions of the second retention mechanism.

In any preceding or subsequent examples, the second portion of the second retention mechanisms may include one or more ball bearings secured within one or more openings in a wall of the housing.

In any preceding or subsequent examples, the ball bearings may be configured to be temporarily positioned within the opening in the wall of the housing for releasably securing of the external objects.

In any preceding or subsequent examples, the quick connect collar mechanism may include a spring configured to apply a pressure on the collar portion to retain the collar portion in a predetermined position to prevent the collar portion from releasing the external objects from within the interior portion of the housing.

In any preceding or subsequent examples, the spring-loaded latches may be rotatable coupled within the interior portion of the housing.

In any preceding or subsequent examples, a latch spring may be configured to be coupled to the spring-loaded latches to apply a pressure on the spring-loaded latches to retain the external objects within the interior portion of the housing and to prevent the spring-loaded latches from releasing the external objects from within the interior portion of the housing and allowing translational movement of the external objects within the interior portion of the housing.

In some examples, the connection mechanism may include an adapter system for a tool, device, etc., such as, for example, an orthopedic impactor or impactor mechanism. The adapter system may include one or more connectors and an adapter mechanism. The adapter mechanism may include a housing having a hollow interior, one or more spring-loaded latches coupled to the housing. The latches may include one or more first retention mechanisms configured to releasably secure the connectors within the hollow interior of the housing and prevent translational motion of the connectors once engaged by the spring-loaded latches. The adapter mechanism may include one or more second retention mechanisms. The connectors may be configured to include one or more adapter locking portions configured to interact with the second retention mechanisms to prevent rotational motion of the connectors once connectors are engaged by the second retention mechanisms.

In any preceding or subsequent examples, the second retention mechanisms may include one or more protrusions and one or more grooves positioned between the protrusions, where, upon interactions of the adapter locking portions of the connectors and the second retention mechanisms, the grooves may be configured to receive the adapter locking portions such that the adapter locking portions are positioned between the protrusions.

In any preceding or subsequent examples, the current subject matter may be configured to include one or more inserts that may be positioned between the adapter mechanism and the connectors.

In any preceding or subsequent examples, the inserts may be configured to be at least partially positioned within an interior portion of the adapter mechanism.

In any preceding or subsequent examples, the first retention mechanisms may include one or more ball bearings secured within one or more openings in a wall of the housing.

In any preceding or subsequent examples, the ball bearings may be configured to be temporarily positioned within the opening in the wall of the housing for releasably securing of the connectors.

In any preceding or subsequent examples, the ball bearings may be configured to be positioned within one or more indentations of the inserts and configured to prevent rotational motion of the connectors once connectors are engaged by the indentations of the inserts.

In any preceding or subsequent examples, the spring-loaded latches may be rotatably coupled within the interior portion of the housing.

Examples of the present disclosure provide numerous advantages. For example, the current subject matter's connection mechanism may be configured to provide for a more secure connection between various tools, instruments, devices, etc. (e.g., a broach and an orthopedic impactor, a drill bit and a power drill, etc.). The connection created by the connection mechanism (e.g., quick connect collar mechanism, adapter mechanism, an insert, etc.) may be configured to prevent and/or limit rotation and/or translation of the connected tools, instruments, devices, etc. once secured. This provides for further stability and ensures safety during operation of the connected tools, instruments, devices, etc. For example, one example implementation of the connection mechanism-the quick connect collar mechanism-provides for fast and easy release of the connected tools, which may be important during operation of the connected tools, such as, for example, when changing between tools, instruments, devices, etc. Additionally, the current subject matter may be configured to provide an ability to utilize existing instrumentation without needing to re-design and recreate specific instrumentation for use with various tools/instruments (e.g., an orthopedic impactor). Further, the current subject matter may also be configured to provide an ability to optimize new connection mechanisms for strength, quality, ease of use, etc. while utilizing existing instrumentation (e.g., the impactor connection to the adapter mechanism may be optimized independently).

Further features and advantages of at least some of the examples of the current subject matter, as well as the structure and operation of various examples of the current subject matter, are described in detail below with reference to the accompanying drawings.

### BRIEF DESCRIPTION OF THE DRAWINGS

The accompanying drawings, which are incorporated in and constitute a part of this specification, show certain features of the subject matter disclosed herein and, together with the description, help explain some of the principles associated with the disclosed implementations. In the drawings, FIG. 1 illustrates a perspective view of an exemplary orthopedic surgical instrument or impactor, in accordance with one or more features of the present disclosure;

FIGS. 2a-h illustrate an exemplary quick connect collar mechanism, in accordance with one or more features of the present disclosure;

FIG. 3 illustrates an exemplary tool connector component, in accordance with one or more features of the present disclosure;

FIG. 4 illustrates an exemplary quick connect collar, in accordance with one or more features of the present disclosure;

FIG. 5a is a perspective view of an example connector system, in accordance with one or more features of the present disclosure;

FIG. 5b is another perspective view of the connector system shown in FIG. 5a, in accordance with one or more features of the present disclosure;

FIG. 5c is another perspective view of the connector system shown in FIG. 5a in an assembled state, in accordance with one or more features of the present disclosure;

FIG. 6a is a perspective view of another example connector system, in accordance with one or more features of the present disclosure;

FIG. 6b is another perspective view of the connector system shown in FIG. 6a, in accordance with one or more features of the present disclosure;

FIG. 6c is another perspective view of the connector system shown in FIG. 6a in an assembled state, in accordance with one or more features of the present disclosure;

FIG. 6d is yet another perspective view of the connector system shown in FIG. 6a in the assembled state, in accordance with one or more features of the present disclosure;

FIG. 7a is a perspective view of another example connector system, in accordance with one or more features of the present disclosure;

FIG. 7b is another perspective view of the connector system shown in FIG. 7a in an assembled state, in accordance with one or more features of the present disclosure;

FIG. 7c is a cut-away section view of the connector system shown in FIG. 7a, in accordance with one or more features of the present disclosure;

FIG. 8a is a block diagram of an exemplary orthopedic surgical instrument and/or impactor in accordance with one or more features of the present disclosure;

FIG. 8b is a flowchart of an exemplary process for operating of the orthopedic surgical instrument and/or impactor shown in FIG. 8a, in accordance with one or more features of the present disclosure;

FIG. 9 illustrates an exemplary computing apparatus, in accordance with one or more features of the present disclosure;

FIG. 10 illustrates an example of a storage medium to store impactor logic, in accordance with one or more features of the present disclosure; and

FIG. 11 illustrates an example computing platform, in accordance with one or more features of the present disclosure.

It should be understood that the drawings are not necessarily to scale and that the disclosed examples are sometimes illustrated diagrammatically and/or in partial views. In certain instances, details that are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

To address these and potentially other deficiencies of currently available solutions, one or more implementations of the current subject matter relate to methods, systems, articles of manufacture, and the like that can, among other possible advantages, provide connection mechanisms, systems, and/or methods that may be used coupling of a tool, device, instrument, etc., such as, for example, but is not limited to, to an orthopedic surgical instrument or impactor.

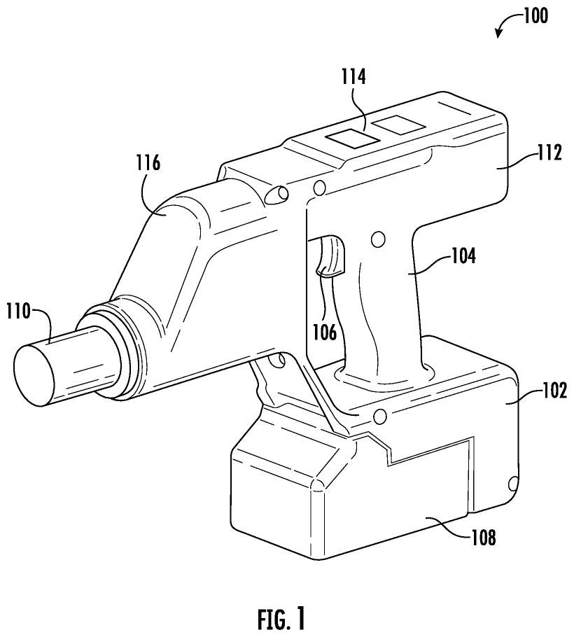

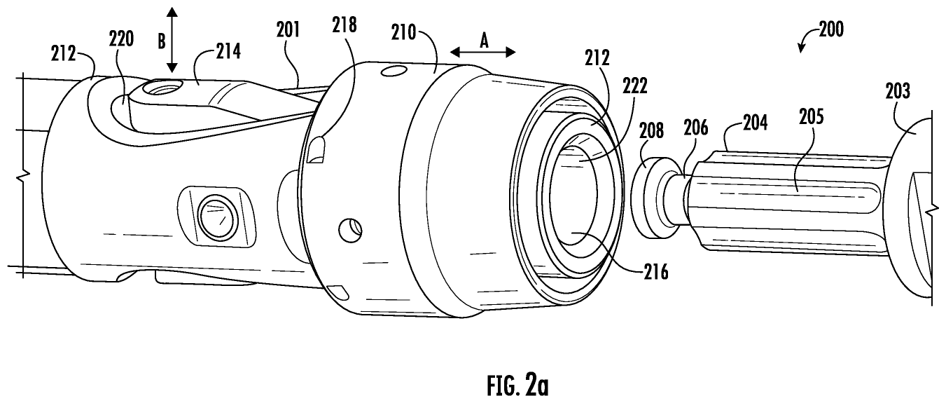

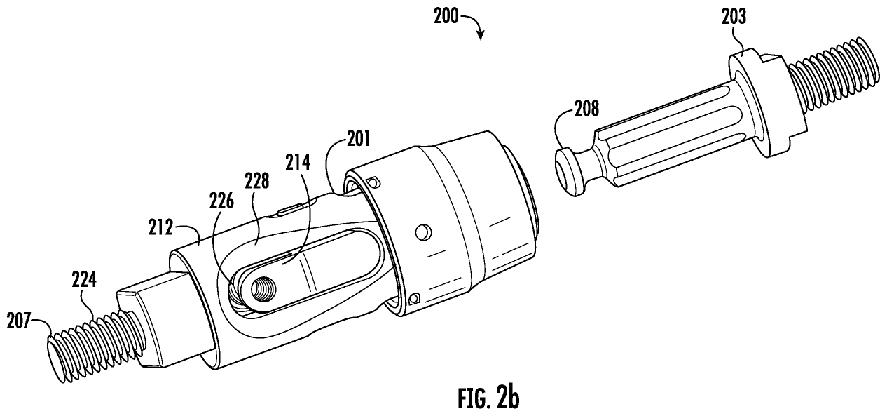

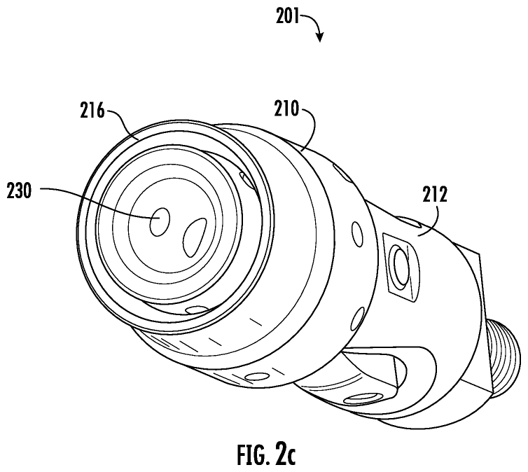

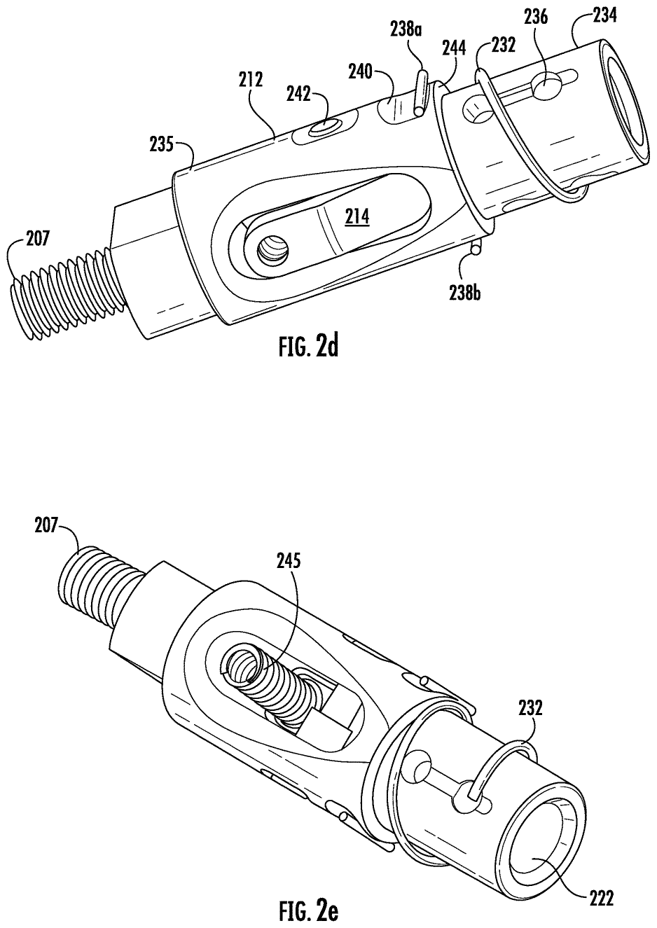

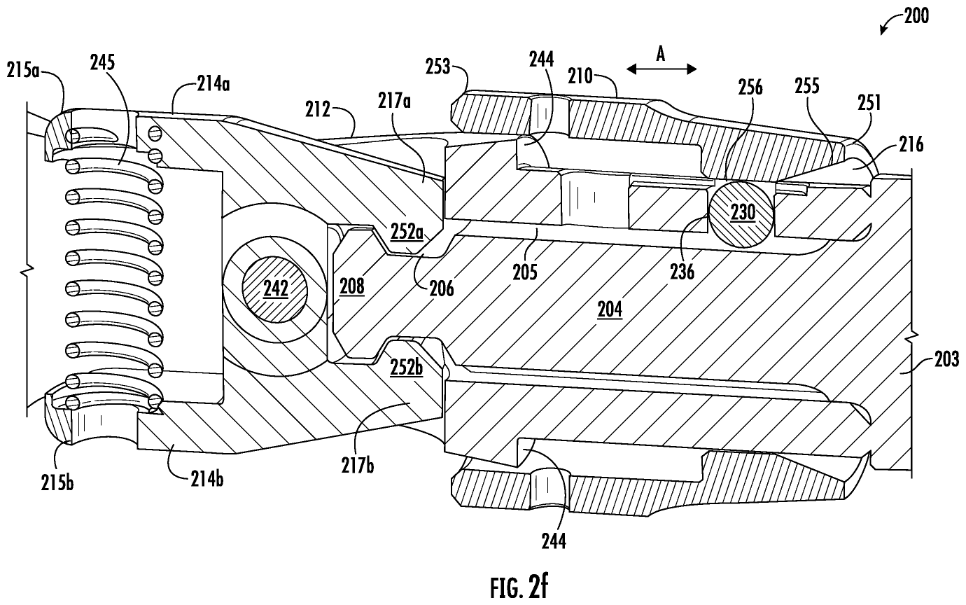

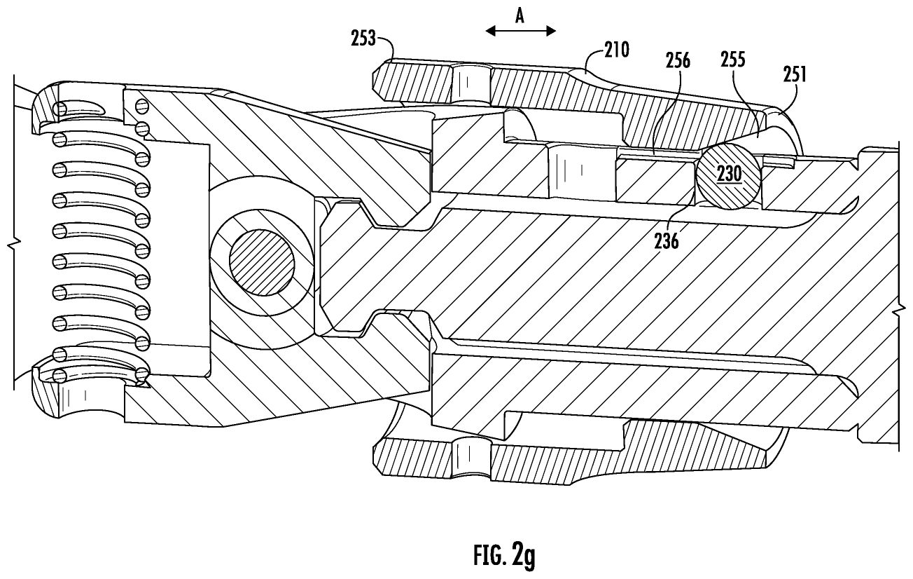

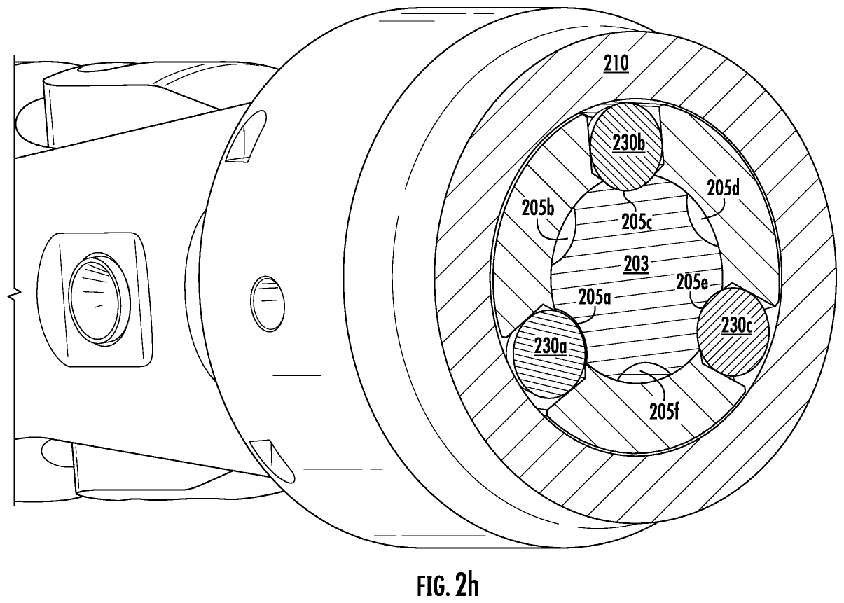

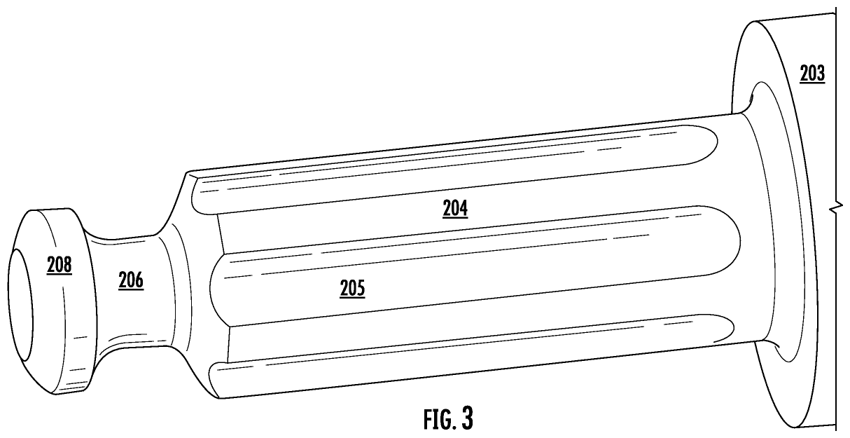

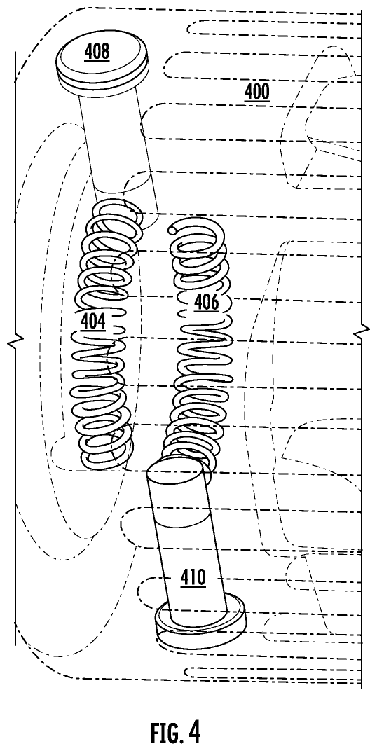

It should be appreciated that while, for example, the connection mechanism(s), system(s), and/or method(s) in accordance with the present disclosure may be described herein in connection with an orthopedic surgical impactor (e.g., as shown in FIG. 1) used to, for instance, drive a broach into a patient's bone to, for example, prepare an intramedullary canal of the patient's bone, the present disclosure is not so limited and the connection mechanism(s), system(s), and/or method(s) (as shown and described below with regard to FIGS. 2a-7c) may be used in connection with any tool, device, instrument, etc. now known or hereafter developed such as, for example, a tap used to insert a bone screw, construction hand tools or power tools, etc. As such, the present disclosure should not be limited to any particular tool unless explicitly claimed.

### I. Orthopedic Impactor

The following description of an orthopedic impactor (e.g., as shown in and discussed in connection with FIGS. 1 and 8a-11) is provided here for exemplary, illustrative purposes only and is not intended to limit the current subject matter and/or any of its elements, applications and/or advantages.

With reference to FIG. 1, an example of an orthopedic surgical instrument, impactor, or impactor mechanism (terms used interchangeably herein without the intent to limit or distinguish) is disclosed. In use, the orthopedic impactor is arranged and configured to transmit a forward energy or motion (e.g., a striking motion, and/or any other motion) to, for example, drive a surgical tool (e.g., a broach) or implant into a patient's bone, and deliver a reverse energy or motion to, for example, remove a struck or lodged surgical tool (e.g., a broach) or implant from a patient's bone.

Orthopedic impactors may be used to position, insert and/or implant an orthopedic implant such as, for example, but not limited to, an acetabular cup, an intramedullary nail, a femoral hip implant, etc. into a bone matter (e.g., a bone of a patient). Alternatively, or in addition to, the orthopedic impactor may be coupled to a surgical tool such as, for example, but not limited to, a broach, to prepare a bone to receive an orthopedic implant.

The orthopedic impactor may be configured to allow selection between the forward and/or reverse application of energy and/or motion by pushing forward and/or pulling back on the orthopedic impactor. During use, a user (e.g., a medical professional, a doctor, a surgeon, etc.) may push forward on an orthopedic impactor thereby causing a hammer of the impactor to strike a first and/or a forward impaction surface causing the orthopedic impactor to drive an orthopedic implant and/or surgical tool. Alternatively, or in addition, the user may pull back on the orthopedic impactor thereby causing the impactor's hammer to strike a second and/or a reverse impaction surface causing the orthopedic impactor to produce a reverse impaction to, for example, remove an orthopedic implant or surgical tool.

It should be appreciated that while, for example, the orthopedic impactor may be described herein in connection with driving a broach into a patient's bone to, for example, prepare an intramedullary canal of the patient's bone, the current subject matter is not so limited, and the orthopedic impactor may be used in connection with any surgical tool and/or implant now known and/or hereafter developed. As such, the current subject matter should not be limited to any particular surgical tool, device, instrument, implant, and/or procedure unless explicitly claimed.

The orthopedic impactor may be arranged and/or configured to accurately and safely cause application of force to an orthopedic implant and/or a surgical tool such as, for example, a broach to prepare an intramedullary canal of a patient's bone and/or to assist with removal of the broach from the intramedullary canal of the patient's bone. The orthopedic impactor may be arranged and/or configured to apply a force (e.g., to deliver a force toward and/or away from a surgical area), while minimizing the risk of injury to the patient and/or to the user's hands during use.

FIG. 1 illustrates an exemplary impactor 100 that may be configured to incorporate a connection system (as shown and described below with regard to FIGS. 2a-7c), according to some implementations of the current subject matter. The impactor 100 may be configured to include a housing 102, a handle 104, a trigger assembly 106, a battery 108, a distal connector assembly 110, a motor housing 112 (that may enclose or house a direct current (DC) motor), an electronic display assembly 114, and a distal connector housing 116. The housing 102 may also support any other appropriate components, tools, instruments, etc., such as, for example, but not limited to, sensors, gauges, bone density instruments, feedback components, or any combination thereof. The housing 102 may also feature one or more connection features that can enable the housing to cooperate with and be used in connection with a robotic surgery system. The connection features may be mounting features, input ports for receiving directional instructions, or any combination thereof.

The housing 102 may be configured to house and/or enclose one or more components of the impactor 100. The housing 102 may be manufactured from any suitable material now known or hereafter developed such as, for example, but not limited to, plastic, metal, composite material, fiberglass, and/or any combination thereof. The housing may be designed to be autoclavable and/or, otherwise, sterilizable using any appropriate method.

The housing 102 may include the handle portion 104 with an optional handgrip for comfortable and secure holding of the impactor 100 for use during a procedure (e.g., positioning of an implant into a bone). Alternatively, or in addition to, the housing 102 may incorporate a suitable mounting interface for integrating the impactor 100 into a robotic assembly during use. In some example implementations, the housing 102 may be a unitary structure and/or may include multiple components that may be assembled together.

The housing 102 may also include a reception port for receiving the battery 108. The battery 108 may be a rechargeable battery and may be removed from the housing 102 after use, such as, for example, for recharging. As can be understood, the battery 108 may recharged while coupled to the housing 102. Alternatively, or in addition, the battery may be integrally formed in the housing and rechargeable through the housing. Use of the battery 108 may provide for portability and versatility of the impactor 100 during use, i.e., the user of the impactor 100 does not have to be concerned with power wires (and/or pneumatic tubes) extending from the impactor 100. Alternatively, or in addition, the housing 102 may include one or more power ports (not shown in FIG. 1) that may be used to couple one or more power wires (e.g., to provide power and/or power in addition to the battery power 108) and/or one or more pneumatic tubes (e.g., to provide additional air pressure to the impactor 100 during use). As can be understood, more than one battery 108 may be included in the housing 102 and/or used during procedures. Any type of battery may be used, such as, for example, but not limited to, alkaline, nickel metal hydride (NiMH), lithium ion, and/or any combination thereof. The battery may also be replaceable and/or rechargeable.

The housing 102 may further be configured to include the motor housing 112 that may enclose a DC motor and/or any other type of motor (not shown in FIG. 1) for operation of the impactor 100. The motor may be configured to cause application of a forward movement (e.g., during an implantation procedure) and/or a reverse movement (e.g., during an implant, tool, etc. removal procedure). Alternatively, or in addition to, more than one motor may be included in the housing 102 for performing different operations. For example, one motor may be used for forward operation of the impactor, while another motor may be used for reverse operation of the impactor.

The motor may be actuated using the trigger assembly 106. As can be understood, actuation of the trigger assembly 106 (e.g., depressing the trigger) may actuate the motor (e.g., motor performing rotary movement). Release of the trigger assembly may stop operation of the motor. During use of the impactor 100, a user (e.g., a medical profession, a doctor, a surgeon, etc.) may hold the impactor 100 with their hand using the handle 104 and position one or more of their fingers on the trigger assembly 106, and when they are ready, depress the trigger assembly 106 to begin performing one or more stages of the surgical procedure.

The housing 102 may include the electronic display assembly 114 that may be used to display one or more operating parameters of the impactor 100. These may include, for example, operating power level, power output level, battery level, direction of operation, etc. The assembly 114 may also display one or more alerts to the user, e.g., a fault in the operation, a low power level, etc. Alternatively, or in addition, the electronic display assembly 114 may be optional.

The housing 102 may further include a distal connector housing 116 that may be configured to house the distal connector 110. The distal connector 110 may be configured to have any desired shape (for illustrative, non-limiting purposes only, a cylindrical shape distal connector 110 is shown in FIG. 1). The distal connector 110 may be used for coupling of various tools, instruments, devices, etc. (e.g., drills, cutting tools, effectors, broaches, implants, etc.) that may be used during a procedure. In some examples, non-limiting implementations, the distal connector may include a coupling mechanism for coupling such tools, instruments, devices, etc. For example, the coupling mechanism may include a quick-connect mechanism to facilitate positioning, exchanges, etc. of tools, instruments, devices, implants, etc. Alternatively, or in addition to, the coupling mechanism may selectively couple to an adapter, which may, in turn, couple to one or more tools, instruments, devices, etc.

### II. Quick Connect Collar Mechanism

During orthopedic surgery, there is often a need to connect two instruments (and/or tools) together using a connection mechanism. Existing connections are overly complex in their operation due to strength and/or safety requirements (e.g., accidental disengagement and/or other fail-safes) and, thus, can be frustrating for the medical professional performing the surgery (or any other user). For example, such connection mechanisms can slow down the surgery, thereby adding time under anesthesia for the patient, create potentially hazardous environment if the connection is unsafe due to its complexity, as well as cause various other problems in the operating room. Further, existing connection mechanisms typically require more hands than are available to the medical professional to operate the mechanism, thus, making them unfavorable and less intuitive, more cumbersome and time consuming (e.g., they add more operating time, create a potential for mis-handling, including instrument dropping, and thereby violating a sterile barrier, etc.). Another problem with existing connection mechanisms is a requirement to completely disconnect two ends of the connection mechanism to be able to change an orientation of a tool being connected between its two sides. Again, this adds time, requires two hands, and increases chances that an instrument drop and/or a violation of sterile field can occur.

Existing connection mechanisms typically utilize a round shaft with a groove cut at some axial location along the shaft. The round shaft usually has one and/or multiple flats and/or keyways cut into it to allow the connection mechanism to control rotation and orientation of the instrument. Examples of this type of existing connection mechanism include drills, taps, and/or other instruments to be manipulated by hand and/or use of a powered instrument, such as, a drill and/or powered impactor device.

Some existing connection mechanisms include quick-connect mechanism that include a spring loaded collar that has a smaller diameter and that radially constrains one or more ball bearings. The collar has a second position and a second larger diameter or tapered surface that allows the ball bearings to move radially outward. To engage, the collar is pulled backward, the mating (e.g., male) component is pushed into the mechanism, until a circumferential groove matches the axial position of the ball bearings. When the collar is released, the ball bearings are pushed inward into the groove of the mating component. Since the collar blocks the expansion path of the ball bearings, the male and female components are locked together. If flats or keyways are cut into the male and female components at matching intervals, a matching number of orientations can be defined. To change orientation, the user needs to pull the collar, remove the male shaft from the female component, rotate either component to the desired orientation, then reconnect the two components.

Some existing connection mechanisms use splined shafts (e.g., for power transmission, which may be common in automotive, industrial and/or agricultural applications). While these shafts and couplings are similar to quick connect mechanisms, instead of flats for orientation control, the mechanisms use splines of common size and pitch. Depending on the application, the shafts can be floating (e.g., not directly retained axially, and held in place by another part of a system) and/or directly axially retained by a snap ring, and/or other quick connect type collar with ball bearings. To change orientation, the user needs to pull the collar and/or release mechanism, remove the male shaft from the female component, rotate either component to the desired orientation, then reconnect the two components.

In some examples, the current subject matter may be configured to relate to a quick-connect collar mechanism that may be used for connection of one or more tools and may be used with an impactor (e.g., as shown in FIG. 1). Some of the features of the quick-connect collar mechanism may include one or more spring-loaded latches and one or more mating groove(s), one or more spring-loaded collar-constraining and freeing ball bearings, and one or more patterned cuts about a common axis that may be configured to extend a certain length along the axis. The mechanism may also include one or more spring-loaded latches and one or more grooves for axial retention of a tool, instrument, etc.

The above latches and/or grooves may be configured to provide no rotational control and/or resistance on their own. For rotational control, the mechanism may include one or more (e.g., three) ball bearings that may be equally-spaced about a common axis of a female component of the mechanism and may be housed in corresponding drill openings. This may allow the ball bearings to radially contract and expand in position with respect to the female component.

The mechanism may also include a spring-loaded collar that may be configured to block and/or allow positional expansion of the ball bearings. The ball bearings may be seated in an axial, rounded grooves that may be patterned around the male shaft axis. When the collar of the mechanism is in a locked position and the ball bearings are constrained, each ball bearing may be seated in a matching axial groove to rotationally lock components together. When the collar is pulled (and/or pushed), the ball bearings may be free to expand to a larger chamber of the collar and the male shaft may be allowed to rotate within the female component until the collar is released, at which point, the ball bearings may be forced into the closest groove. If the latches are not depressed, the male shaft may be axially locked within the female component and the two cannot be axially separated. This may enable an independent control of axial connection and rotational orientation.

FIGS. 2a-2h illustrate an exemplary quick-connect collar system 200, according to some examples. The system 200 may include a quick connect mechanism 201 and a tool connector component 203. The tool connector component 204 may be configured to be inserted and secured within the quick connect mechanism 201.

As shown in FIGS. 2a-c and FIG. 3, the tool connector component 203 may include a body 204 having a distal end 208. A latch mating groove 206 may be disposed proximate to the distal end 208 and may be substantially perpendicular to a longitudinal center axis of the body 204. The groove 206 may be configured for use during a secure coupling of the tool connector component 203 to the quick connect mechanism 201 upon insertion of the tool connector component 203 into the quick connect mechanism 201.

The body 204 may also include one or more ball bearing mating grooves or trenches 205. The grooves 205 may be disposed along the length (or substantially parallel to the longitudinal center axis) of the body 204. The depth of the grooves 205 may be such that they are able to accommodate positioning of the ball bearings 230 (not shown in FIGS. 2a-b; the ball bearings 230 are shown in FIG. 2c) of the quick connect mechanism 201. As can be understood, any configuration of the tool connector component 203 is possible and the current subject matter is not limited to the shown configuration of the component 203 (as well as the one shown in FIG. 3).

The quick connect mechanism 201 may include a quick connect collar 210, an inner sleeve housing 212 having a hollow housing 222 and a tool connector receiving opening 216, one or more latches 214, and one or more openings 218 of a collar stopping mechanism. The hollow housing 222 and the tool connector receiving opening 216 may have diameters that may be greater than the diameter of the body 204 of the tool connector component 203. This may allow the body 204 of the tool connector component 203 to be inserted and securely coupled within the hollow housing 222. Secure coupling of the tool connector component 203 within the hollow housing 222 may be accomplished using the latches 214 and the collar 210 that may be configured to prevent translational and rotational movements, respectively, of the tool connector component 203 once the latches 214 and the collar 210 are separately engaged. Translational movement of the tool connector component 203 may be accomplished upon the latches 214 engaging with the latch mating groove 206 of the tool connector component 203. Rotational movement of the tool connector component 203 may be accomplished upon one or more ball bearings 230 engaging with one or more ball bearing grooves 205 of the tool connector component 203.

To allow for rotational movement of the tool connector component 203 (while preventing its translational movement), the collar 210 may be disengaged from the body 204 of the tool connector component 203, for example, by the user pulling the collar 210 away from opening 216 (in a direction A, as shown in FIG. 2a). The movement of the collar 210 releases the ball bearings 230 from the grooves 205, thereby allowing the tool connector component 203 to rotate within the hollow interior 222 of the housing 212. To re-engage, the collar 210 may be released toward the opening 216, thereby engaging the ball bearings 230 with the grooves 205. In some examples, the collar 210 may include a spring-loaded mechanism (e.g., a spring) (not shown in FIGS. 2a-b) that may be configured to push the collar 210 toward the opening 216 upon the user releasing it from being held away from the opening 216.

To allow for translational movement of the tool connector component 203 (while preventing it rotational movement), the latches 214 (which may also be spring-loaded) may be pressed toward the interior of the housing 212 (e.g., in a direction B, as shown in FIG. 2a), thereby disengaging them from the latch mating groove 206. While the latches 214 are disengaged from the latch mating groove 206, the ball bearings may still be engaged within the ball bearing grooves 205. This may allow movement of the tool connector component 203 in and out of the hollow interior 222 (e.g., the tool connector component 203 may be removed in its entirety from the hollow interior 222.

In some examples, to allow for both the translational and rotational movement of the tool connector component 203 within the hollow interior 222, both the latches 214 may be pressed toward the interior of the housing 212 and the collar 210 may be pushed away from the opening 216. Such movements may fully release the tool connector component 203 from the hollow interior 222 of the housing 212.

As shown in FIGS. 2a-b, the latches 214 may be configured to be positioned within one or more corresponding cavities 226 in the housing 212. The cavities 226 may also include corresponding openings 228 that may be sized to accommodate movement of latches 214 in and out of the interior of the housing 212. The cavities 226 and the openings 228 may be configured to prevent extension of latches 214 beyond the surface of the housing 212. This may allow the collar 210 to freely slide along the housing 212.

In some examples, the collar 210 may also incorporate one or more openings 218 of a stopping mechanism that may be configured to mechanically stop movement of the collar 210 beyond a predetermined stopping point on the housing 212. This may prevent the collar 210 from contacting the latches 214 and potentially, being stuck on the latches 214 when pulled back away from the opening 216 too far.

The housing 212 may also include a distal end 207 that may include a connector 224. The connector 224 may be configured to be coupled to an operating tool, such as the impactor 100 shown in FIG. 1. The connector 224 may be a threaded connector that may allow the mechanism 201 to be threaded into an appropriate threaded receptacle in the impactor, such as in its distal connector 110, as shown in FIG. 1, and/or any other tool.

FIGS. 2d-e illustrate the quick connect mechanism 201 shown in FIGS. 2a-c but with the collar 210 removed to illustrate some of the interior portions of the mechanism 201. In particular, FIG. 2e shows the quick connect mechanism 201 with the latches 214 removed.

As shown in FIGS. 2d-e, the quick connect mechanism 201 may include a collar spring 232 that may be configured to be wrap around a collar positioning portion 234 of the housing 212. The collar positioning portion 234 may be configured to have smaller dimensions (e.g., a smaller diameter) than the remaining portion 235 of the housing 212, thereby creating a ledge 244. The collar spring 232 may be configured to be secured to the ledge 244 of the housing 212 and extend from the ledge 244 to substantially the opening 216. By extending around the collar positioning portion 234, the spring 232 may be configured to interact with an interior portion of the collar 210 (not shown in FIGS. 2d-e), thereby creating a spring-loaded tension to force the collar 210 to be normally pushed toward the opening 216.

The portion 236 may also include one or more ball bearing openings 236 that may be configured to accommodate positioning of one or more ball bearings 230 (not shown in FIGS. 2d-e). The openings 236 may be sized to allow the ball bearings 230 to extend into the hollow interior 222 so that they can interact with the grooves 205 of the component 203 while at the same time preventing the ball bearings 230 from falling through into the hollow interior 222. The collar 210 may be configured to secure the ball bearings 230 on an exterior surface of the portion 234 to prevent the ball bearings 230 from falling out in opposite direction (of the interior 222).

As discussed above, movement of the collar 210 along direction A (as shown in FIGS. 2a-c), may be configured to engage (when the collar 210 is translated toward the opening 216) and disengage (when the collar 210 is translated away from the opening 216) the ball bearings 230 located within the openings 236.

The portion 235 of the housing 212 may also include one or more indentations 240 and one or more rods 238(a, b), which, collectively form one or more stopping mechanisms. The rods 238 may be configured to be rotatably secured within the openings 218 of the collar 210 (as shown in FIGS. 2a-c). Upon movement of the collar 210 along the length of the housing 212, the rods 238 may be configured to move within the respective indentations 240 and prevent overextension of the collar 210 in either direction.

The latches 214 may be configured to be pivotally coupled about a rod 242 that may be secured to the housing 212 perpendicular to the axis of movement of the collar 210 (e.g., perpendicular to the direction A, as shown in FIGS. 2a-c). The pivotal coupling of the latches 214 may be configured to allow partially-rotational movement of the latches 214 about the rod 242 to allow the latches 214 to create a locking arrangement with the groove 206 of the tool connection component 203. The latches 214 may also be coupled to a spring 245 (e.g., both latches 214 may be coupled to the spring 245). The spring 245 may be configured to create a tension between latches 214, thereby forcing the latches 214 to be automatically engaged in a latching position (e.g., a default position) with the groove 206. To release the latches 214 from the groove 206, pressure may be applied to the latches 214 against the tension of the spring 245 to rotate the latches about the rod 242 away from the groove 206.

FIGS. 2f-g are cross-sectional views of the quick-connect collar system 200, and in particular, of the system 200 with the quick connect mechanism 201 being engaged with the tool connector component 203. FIG. 2f illustrates the tool connector mechanism 203 being rotationally and translationally engaged with the quick connect mechanism 201, whereas FIG. 2g illustrates the tool connector mechanism 203 being translationally engaged but rotationally disengaged with the tool connector mechanism 203. FIG. 2h illustrates a cross-sectional view of ball bearings 230 engaging with the tool connector mechanism 203.

Referring to FIGS. 2f-g, each latch 214(a, b) may include respective proximal ends 215(a, b) and distal ends 217(a, b). The proximal ends 215 may be configured to be coupled to the spring 245. The distal ends 217 may be configured to include respective tool connector engagement teeth 252(a, b). The tool connector engagement teeth 252 may be configured to be interact or engage with the groove 206 of the tool connector mechanism 203 to lock the tool connector mechanism 203 within the hollow interior 222 of the quick connect mechanism 201 and prevent translational movement of the quick connect mechanism 201 during use.

To release the tool connector component 203 and allow its translational movement within/from the hollow interior 222 of the housing 212, pressure may be applied to the proximal ends 215 of the latches 214 (e.g., by squeezing the ends 215 together), which causes compression of the spring 245 and bringing of the ends 215 closer together. Because of the pivotal coupling of the latches 214 to the rod 242, application of pressure to the proximal ends 215 also causes rotation of the latches 214 about the rod 242 and removal of the teeth 252 from the groove 206 of the tool connector component 203, thereby releasing the tool connector 203 for translational movement.

To engage the tool connector component 203, the above process may be performed in reverse. Alternatively, or in addition, the tool connector component 203 may be forcefully inserted into the hollow interior 222 and pushed against the teeth 252 causing them to spread apart to allow further protrusion of the tool connector component 203 inside the hollow interior 222 until the teeth 252 snap into the groove 206 of the tool connector component 203. The teeth 252 may include respective slanted edges 257(a, b) that may assist with this engagement. The slanted edges 257 may have a predetermined angle that may allow relative ease of insertion of the tool connector component 203.

As shown in FIGS. 2f-g, the collar 210 may include a proximal end 253 and a distal end 251. The proximal end 253 may be disposed proximate to the latches 214 and the distal end 251 may be disposed proximate to the opening 216. The collar 210 may be moved between a locked or engaged position (shown in FIG. 2f) and an unlocked or disengaged position (shown in FIG. 2g). In the engaged position, the collar 210, using its flat edge 256, may be configured to push one or more ball bearings 230 positioned inside their respective ball bearing openings 236 into grooves 205. In the engaged position, the collar 210 along with the ball bearings 236 being engaged within the grooves 205 may be configured to prevent rotational motion of the tool connector component 203.

To allow for rotational motion of the tool connector component 203, the collar 210 may be pulled away from the opening 216, as shown in FIG. 2g. By pulling the collar 210, the flat edge 256 may be configured to disengaged from the ball bearing 230, thereby allowing the ball bearing 230 to move inside its opening 236 and away from the tool connector component 203. Such movement may be assisted by a slanted edge 255 of the collar 210. The slanted edge 255 may be adjacent to the flat edge 256 and positioned proximate to the distal end 251. In particular, the edge 255 may be slanted at an angle away from the flat edge 256 and toward the distal end 251. The slanted edge 255 may be so structured to prevent the ball bearing 230 from entirely falling out of its opening 236 once the collar 210 is moved into the disengaged position shown in FIG. 2g.

FIG. 2h illustrates the tool connector component 203 being engaged by the ball bearings 230(a, b, c) within its grooves 205(a, c, e), respectively. As shown in FIG. 2h, the tool connector component 203 may include six (6) grooves 205(a, b, c, d, e, f) and the quick connect mechanism 201 may include three (3) ball bearings 230(a, b, c). Use of a smaller number of ball bearings 230 may allow the user to temporarily disengage the tool connector component 203 (by pulling on the cover 210, as shown in FIG. 2g) so that it may be rotated to a desired position and then engage the ball bearings 230 within a different set of grooves 205 (e.g., grooves 205(b, d, f)) by releasing the collar 210, as shown in FIG. 2f.

As can be understood, there can be any number of ball bearings 230 and corresponding grooves 205. In some exemplary, non-limiting implementations, the number of ball bearings 230 may be the same or different from the number of grooves 205. Moreover, grooves 205 may be uniformly spaced out around an exterior circumference of the body 204 of the tool connector component 203 (e.g., the grooves 205 may be equidistant from one another around the circumference of the body 204). As can be understood, grooves 205 may be randomly positioned on the tool connector component 203.

Further, more or less ball bearings 230 may be used by increasing and/or decreasing diameters of the body 204 and/or housing 212 and/or ball bearing sizes (e.g., more ball bearings may be fitted with a smaller ball bearing size and/or larger male/female diameters and vice versa). The ball bearing positioning in the housing 212 (and/or in the interior 222) may be configured to match the groove 205 pattern of the component 203 such that the desired rotational orientations may be configured to align and each ball bearing 230 may always have a groove 205 to seat into.

Referring to FIG. 3, which illustrates the tool connector 203, the distal end 208 may have a smaller diameter and/or size than the diameter and/or size of a circumference created by a bottom edges of the grooves 205 (e.g., tangent points of the ball bearings 230). This is by design so that the user can simply push the two pieces together and allow the ball bearings to engage directly into the axial grooves. If the latch feature was large in diameter than the inscribed circle of the ball bearings, the user would have to pull the spring-loaded collar back to allow the ball bearings to expand and ride over this feature.

As shown in FIGS. 2a-c and FIG. 3, the tool connector component 203 may include a body 204 having a distal end 208. A latch mating groove 206 may be disposed proximate to the distal end 208 and may be substantially perpendicular to a longitudinal center axis of the body 204. The groove 206 may be configured for use during a secure coupling of the tool connector component 203 to the quick connect mechanism 201 upon insertion of the tool connector component 203 into the quick connect mechanism 201.

In some examples, actuation of the collar 210 does not hinder depression of the latches 214. For example, actuation of the collar 210 may be configured to block the latches from opening (e.g., additional material may be added at one or more outer surfaces of the latches). This may be configured to act as a “safety by design” feature to ensure that the rotational lock may be actuated without compromising the strength of the translational lock.

Further, actuation of the collar 210 may be configured to not cause actuation of the latches 210. In some examples, actuation of the collar 210 may depress the latches 214 (e.g., by way of extending the collar 210 and adding a ramped surface that may depress the latches 214 as the collar 210 is pulled towards the latches 214). This may be beneficial if a simpler function is desired, and/or if a collar-only design is desired. For example, the collar 210 may be a two-stage and/or two-step locking collar, where in a first stage and/or first axial position of the collar 210, the ball bearings 230 may be released but not yet come into contact with spring-loaded levers. In a second stage and/or second axial position of the collar 210, the collar 210 may be configured to come into contact with the latches 214, thereby releasing the translational lock. The second stage may be used to either maintain the release of the rotational lock of the ball bearings 230 by having a larger diameter chamber, and/or the internal geometry may be such that it may taper back down to a smaller diameter re-locking the ball bearings 230 to retain an independent locking control. This may be beneficial if the desired function is to have independent locking control but to only have a collar-actuated mechanism.

In some examples, while the above process operates with spring loaded latches 214, the translational engagement/lock may also be accomplished using a spring loaded button, a single latch, a spring loaded lever, a cross pin, a snap ring, a retaining clip, a cam actuated by a rotating collar and/or lever, another spring loaded collar, and/or it may be configured to release as a result of a second stage of the described spring loaded collar (e.g., in a first position, the rotational lock is released, and in a second position, the latches 214 are released).

In some examples, while the collar 210, as described above, may be configured to translate towards the latches 214 for disengagement of the ball bearings 230, the collar 210 may be designed to move in an opposite direction for disengagement of the ball bearings 230.

In some examples, while the collar 210, as described above, may be configured to translate axially between the engaged/locked and disengaged/unlocked positions, it may also be configured to rotate into one or more pre-determined engaged/locked and disengaged/unlocked positions. For example, instead of having smaller diameter and larger diameter portions to allow and block expansion, the collar 210 may be configured to have one or more matching axial grooves cut into it to allow the expansion of the ball bearing 230 into these grooves. To enable this, a cap may be configured to be provided and rotationally spring-loaded via a torsional spring. Alternatively, or in addition, small springs 404, 406 may be configured to be wrapped around the diameter of the housing and actuated via corresponding cross pins 408, 410 mounted on the collar 400.

### III. Impact Drive Connection Mechanism

As stated above, during orthopedic surgery, there is often a need to connect one or more instruments and/or tools together using a connection mechanism. Connection geometries, associated with such connections, are typically uniquely specific to particular surgical systems that are used during the surgery. This is done to prevent cross-usage of mixed tools of different surgical equipment makers, as well as to make strength and/or other quality improvements to a connection design. In the creation of a universal tool such as a powered impactor that is designed to be used with multiple existing instrument/implant surgical systems (or “families”), these instruments would need to be redesigned and recreated with a common connection mechanism or multiple powered impactor types would need to be created to be system and/or family specific.

In some examples, the current subject matter's connection mechanism may include an adapter that may be positioned between a powered impactor device and any instruments and/or tools. The adapter may be used for accommodating specific connection geometries as well as new and existing connection geometries without recreating instruments/tools and/or the powered impactor. In some examples, a full length adapter provides a male end of one connection and a female end of another connection. Each connection geometry may include various components for retention and/or release and/or anti-rotation.

This disclosure also provides a spacer that may leverage one or more features of an existing connection of an instrument/tool to a connection geometry of a powered impactor. Specifically, the spacer may be configured to re-use a retention feature of any existing connections (e.g., the circumferential groove of a shaft of an instrument/tool/etc.), and may include one or more anti-rotation features at a separate axial location.

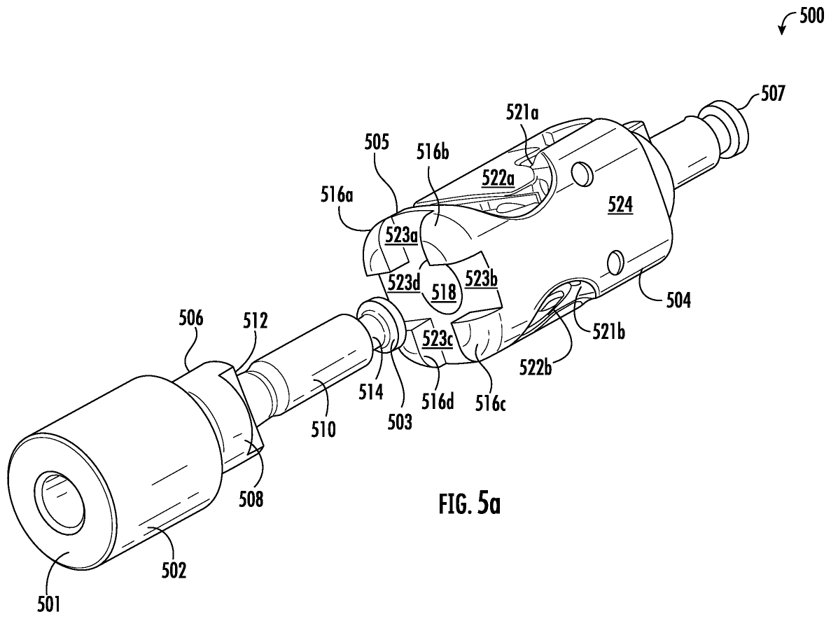

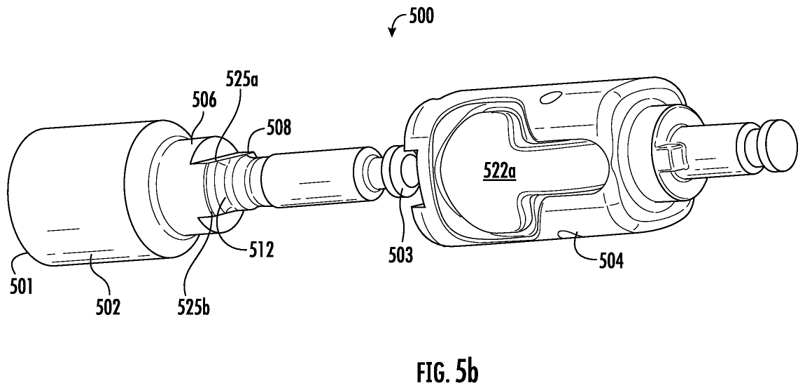

FIGS. 5a-c illustrate an example connector system 500, according to some implementations of the current subject matter. The system 500 may include a connector 502 and an instrument/tool adapter 504. The adapter 504, at its distal end 507, may be configured for connection to an impactor. The connector 502 may be configured to be inserted and secured within the adapter 504. In particular, the adapter 504, at its proximal end 505, may be configured to include an opening 518 that may be used for accommodating insertion of the connector 502 to be secured within an interior portion of the adapter 504.

The connector 502 may include a proximal end 501 and a distal end 503. The proximal end 501 may be configured for coupling to various instruments/tools, etc. (not shown in FIGS. 5a-b). The distal end 503 may be configured for insertion into the opening 518 of the adapter 504. The connector 502 may further include a body 510 coupled to a locking platform 506 having an adapter locking component 508 positioned at a proximal end 512 of the body 510. A latch mating groove 514 may be disposed proximate to the distal end 503 and may be substantially perpendicular to a longitudinal center axis of the body 510. The groove 514 may be configured for use during a secure coupling of the connector 502 within the interior of the adapter 504 upon insertion of the connector 502, through the opening 518, into the adapter 504.

The adapter locking component 508 may be configured to include one or more walls 525(a, b) that may be configured to extend parallel to a longitudinal axis of the body 510 of the connector 502 and may be further configured to extend away from the distal end 512 of the body 510. Moreover, the length (and/or height) of the walls 525 may be configured for fitting within one or more grooves 523(a, b, c, d) and between one or more protrusions 516(a, b, c, d) of the adapter 504, as discussed herein (and shown in FIG. 5c).

The adapter 504 may include a body 524 having the proximal end 505 and the distal end 507. As stated above, the proximal end 505 may be configured to include the opening 518 for receiving the connector 502, and in particular, body 510 of the connector 502. The opening 518 may be sufficiently sized so as to allow insertion of the body 510 into the interior of the body 524 of the adapter 504.

The body 524 of the adapter 504 may be configured to include one or more latches 522(a, b) that may be disposed on opposite sides of the body 524. The latches 522 may be disposed within respective latch cavities 521(a, b) on the body 524 of the adapter 504. The latches 522 may be spring-loaded latches (e.g., with springs disposed within the interior portion of the body 524) that may be used to secure and/or release the body 510 of the connector 502. In one example, the latches 522 may be configured to interact with the groove 514 disposed at the distal end 503 of the connector 502. Upon insertion of the body 510 into the opening 518, the body 510 may be configured to push against the spring-loaded latches 522 until the latches 522 interact with the groove 514 and secure the body 510 of the connector 502 within the interior of the body 524. To remove the connector 502 from the interior of the body 524, the latches 522 may be depressed (e.g., by pushing the latches 522 within respective cavities 521), thereby releasing the body 510 of the connector 502 and allowing removal of the connector 502 from the body 524. While FIGS. 5a-c illustrate use of latches 522 for securing the body 510 in the interior of the adapter 524, the current subject matter is not limited to the use of the latches and, as can be understood, any other mechanisms and/or methods may be used to retain the body 510 in the interior of the adapter 524 and/or restrict its translational movement.

The body 524 may also be configured to include one or more grooves 523(a, b, c, d) and one or more protrusions 516(a, b, c, d) positioned at the proximal end 505 of the body 524. The protrusions 516 may be configured to perpendicularly extend away from the proximal end 505 of the body 524. The grooves 523 and protrusions 516 may be configured to alternate with one another. For example, the groove 523a may be positioned between adjacent protrusions 516a and 516b; the groove 523b may be positioned between protrusions 516b and 516c; the groove 523c may be positioned between protrusions 516c and 516d; and the groove 523d may be positioned between protrusions 516d and 516a.

In some examples, in view of the extending protrusions 516, each groove 523 may be configured to be defined by two substantially vertical walls and a substantially flat bottom portion. Such example geometrical arrangement of the protrusions 516 and the grooves 523 may be configured to allow receiving of the adapter locking component 508. The width of the adapter locking component 508 may be configured to be less than a distance between two walls of protrusions 516 (or, for example, a width of a groove 523).

As shown in FIG. 5c, continuous insertion of the body 510 of the connector 502 into the opening 518 of the adapter 504 may be configured to cause the adapter locking component 508 to be positioned into two opposite grooves 523 and between two sets of protrusions 516. For example, the adapter locking component 508 may be positioned between protrusions 516a and 516b as well as protrusions 516c and 516d (not shown in FIG. 5c). Moreover, the adapter locking component 508 may be settled within the grooves 523a and 523c (not shown in FIG. 5c). As can be understood, the adapter locking component 508 (e.g., by rotating the connector 502 prior to insertion into the adapter 504) may also be positioned between protrusions 516b and 516c as well as protrusions 516a and 516d (not shown in FIG. 5c) and settled within the grooves 523b and 523d (not shown in FIG. 5c). Upon positioning of the adapter locking component 508 between pairs of protrusions 516 and settling it into respective grooves 523, the walls 525 of the adapter locking component 508 may be configured to be positioned against and/or adjacent to the walls of respective protrusions 516.

In some examples, as shown in FIG. 5c, the coupling of the connector 502 and the adapter 504 using the latches 522 and the connector body 510 (and in particular, the groove 514) may be configured to prevent translational movement of the connector 502 and/or the adapter 504 with respect to one another, thereby prohibiting accidental disengagement of any instruments and/or tools that may be coupled to the connector 502 and/or the adapter 504. Moreover, use of the adapter locking component 508 of the connector 502 along with the protrusions 516 and the grooves 523 may be configured to prevent rotational movement of the connector 502 and/or the adapter 504 with respect to one another, thereby prohibiting undesired spinning/rotation of any instruments and/or tools that may be coupled to the connector 502 and/or the adapter 504. As can be understood, there may be any number of protrusions 516 and/or grooves 523 and/or width of the adapter locking component 508 may be appropriately adjusted to accommodate its positioning within the grooves 523. Moreover, securing of the body 510 of the connector 502 within the interior portion of the adapter 504 may be accomplished in any desired way.

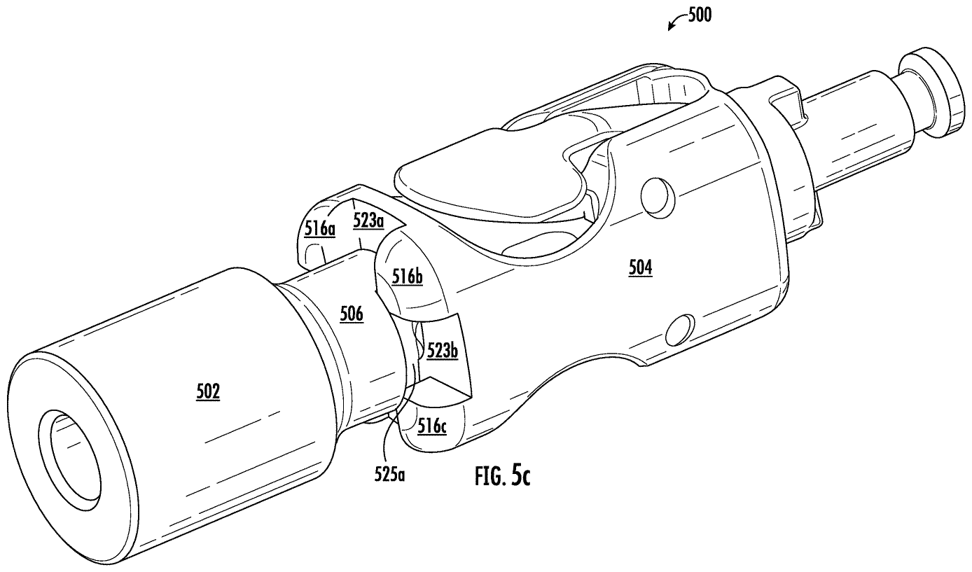

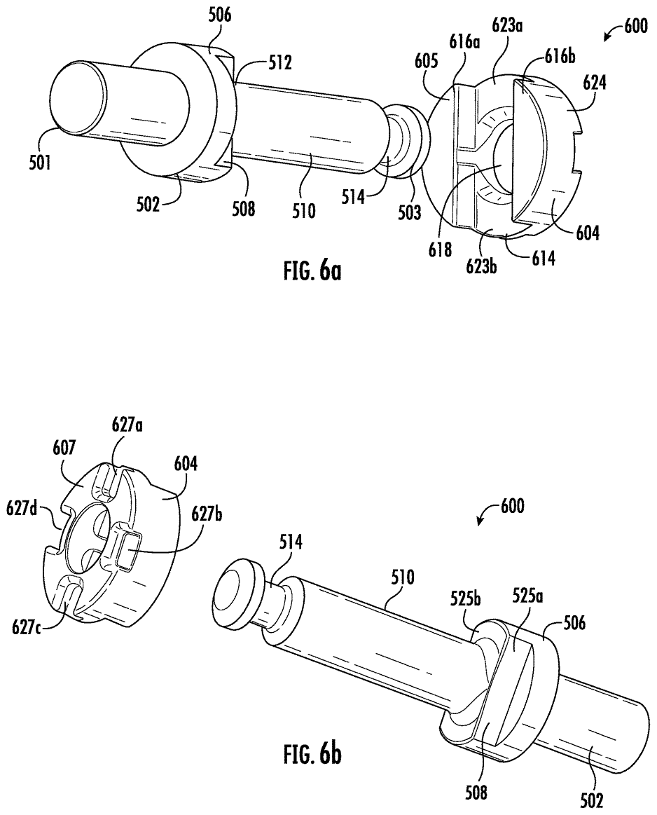

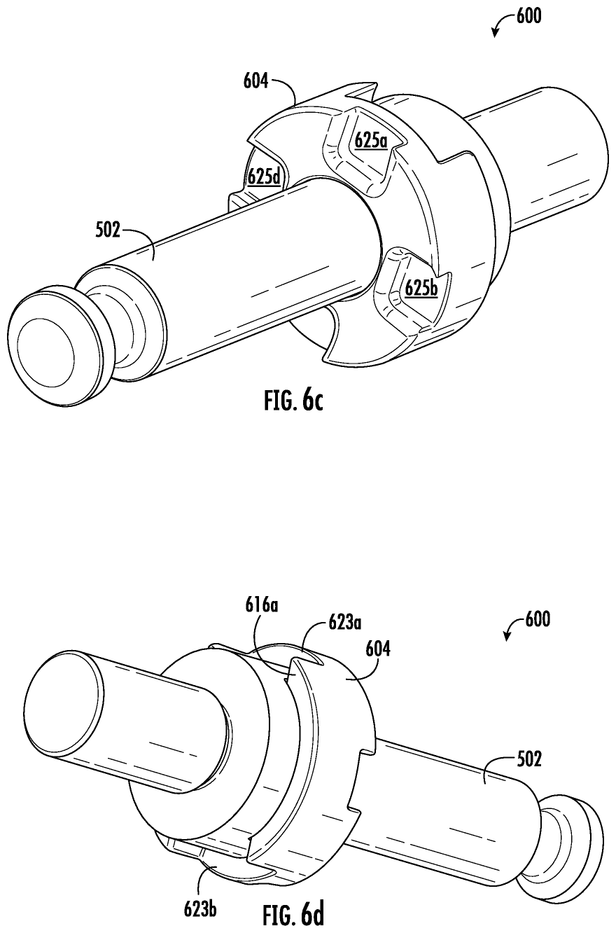

FIGS. 6a-d illustrate another example connector system 600, according to some implementations of the current subject matter. The system 600 may include the connector 502 (as shown in FIGS. 5a-c) and an instrument/tool adapter 604. The adapter 604 is different from the adapter 504 (shown in FIGS. 5a-c) as it includes indentations that may be used for secure positioning and/or mating of tools/instruments on one side and the adapter locking component 508 on the other side.

Referring to FIGS. 6a-b, the adapter 604, at its distal end 607, may, as stated above, be configured for connection to a tool/instrument, e.g., an impactor. The connector 502 may be configured to be inserted through and secured to the adapter 604. In particular, the adapter 604 may be configured to include an opening 618 that may be used for accommodating protrusion of the connector 502 through the adapter 604.

The adapter 604 may include a body 624 having the proximal end 605 and the distal end 607, where the opening 618 may be configured to protrude between the proximal and distal ends 605, 607, respectively. The opening 618 may allow protrusion of the connector 502, and in particular, the body 510 of the connector 502. The opening 618 may be sufficiently sized so as to allow insertion of the body 510 through it.

The groove 614 may be configured to include one or more groove portions 623a, 623b and one or more protrusions 616a, 616b positioned at the proximal end 605 of the body 604. The protrusions 616 may be configured to extend away from the proximal end 605 of the body 604. In one example, the protrusions 616 may be configured to extend substantially perpendicularly away from the proximal end 605. The groove portions 623 may be positioned between the protrusions 616 allowing positioning of the adapter locking component 508 between the protrusions 616 and within the groove portions 623.

As shown in FIGS. 6a-b, the adapter 604 includes two protrusions 616 as compared to the four protrusions 516 shown in FIGS. 5a-c. However, as can be understood, the adapter 604 (or the adapter 504 shown in FIGS. 5a-b) can include any number of protrusions (and/or corresponding grooves) that may be used for securing the adapter and the connector. Because the adapter 604 includes two protrusions 616, two groove portions 623 may be defined by their two vertical walls and a substantially flat bottom portion for securing of the adapter locking component 608 by placing it adjacent the flat bottom portion and against the vertical walls of the groove portions 623.

As stated above, at its distal end 607, the adapter 604 may be configured to include locking indentations 627(a, b, c, d). While four indentations 627 are shown, as can be understood, any number of indentations 627 may be used. The indentations 627 may be used for securing the adapter 604 (with and/or without the connector 502 coupled thereto) to one or more tools and/or instruments (e.g., an impactor tool). For example, one or more such tools/instruments may include one or more teeth and/or extensions that may be configured to mate with the indentations 627 to provide a temporary rotation lock between the tool/instrument and the adapter 604.

The indentations 627 may be configured to be positioned around the circumference of the opening 618. For example, the indentation 627a may be configured to be positioned opposite the indentation 627c, and the indentation 627b may be configured to be positioned opposite the indentation 627d. The indentations 627 may be configured to have a predetermined depth to accommodate secure positioning and/or mating of the teeth of the tools/instruments (e.g., an impactor). As can be understood, any other arrangement, depth, and/or number of the indentations 627 may be possible.

FIGS. 6c-d are perspective views of the system 600 having the connector 502 and the adapter 604 coupled to each other. The coupling of the connector 502 and the adapter 604 may be performed in a way that is similar to the coupling of the connector 502 and the adapter 504 shown in FIGS. 5a-c, where the connector 502's adapter locking component 508 may be positioned between protrusions 616a and 616b and settled in the groove portions 623 of the adapter 604.

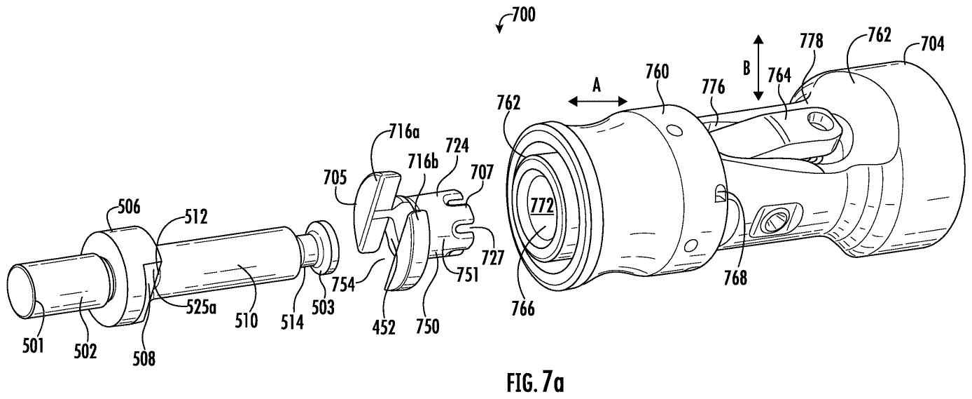

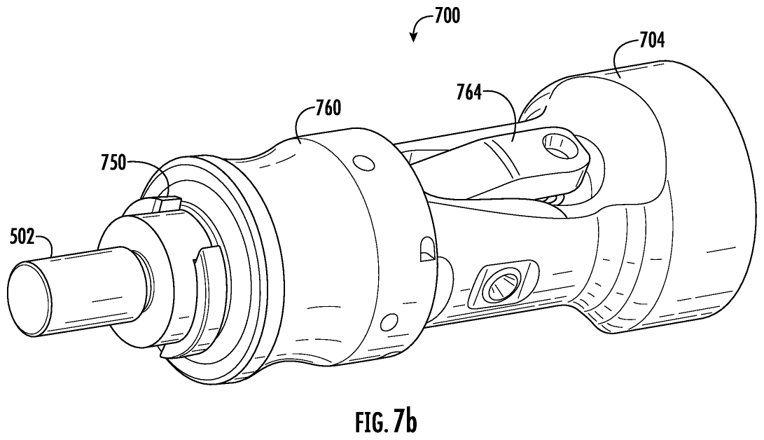

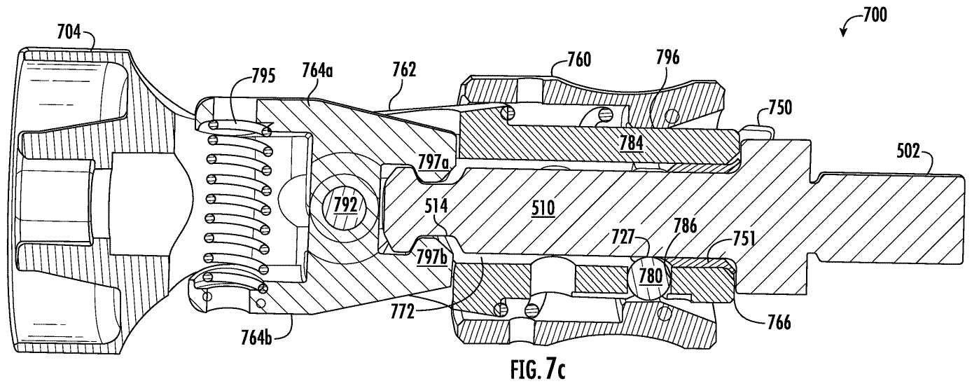

FIGS. 7a-c illustrate another example connector system 700, according to some implementations of the current subject matter. In addition to the connector 502 (as is also shown in FIGS. 5a-c), an instrument/tool adapter 704 (which may be connected to a tool/instrument), the system 700 may include an insert 750 that may be used for securing the connector 502 within the adapter 704 as well as preventing rotational and translational movement of the connector 502 once secured. The connector 502 may be configured to be inserted through and secured to the adapter 704 via the insert 750. In particular, the adapter 704 may include an opening 766 that may be used for accommodating protrusion of the connector 502 and the insert 750 through the adapter 704. The insert 750 may include an insert opening 752 that may allow protrusion of the connector 502, as shown in FIG. 7c.

The insert 750 may include a body 724 having a proximal end 705 and a distal end 707. As stated above, the insert's body 724 may include opening 752 that may be configured to receive the connector's body 510 and may extend between the proximal and distal ends 705, 707, respectively, where the opening 752 may be sufficiently sized so as to allow protrusion of the body 510 through it.

The body 724 may also be configured to include a groove 754 and one or more protrusions 716a, 716b positioned at the proximal end 705 of the body 724, where the protrusions 716 may extend away (e.g., substantially perpendicularly) from the proximal end 705. The groove 754 in the insert 750, defined by vertical walls of the protrusions 716, may be configured to receive the adapter locking component 508 between the protrusions 716. Mating of the adapter locking component 508 and the groove 754 may be configured to constrains and/or prevent rotational movement of the connector 502.

At its distal end 707, the body 724 of the insert 750 may be configured to include a cylindrical extension body 751 that may include one or more ball bearing locking indentations 727 disposed proximate to the distal end 707. The ball bearing locking indentations 727 may be used for interaction with one or more ball bearings positioned in the interior portion of the adapter 704. Any number of indentations 727 may be used. The indentations 727 may be used for securing the insert 750 in the interior portion of the adapter 704 and preventing rotational movement of the insert 750 (and thus, the connector 502, if coupled thereto).

The indentations 727 may be configured to be positioned around the circumference of the extension body 751 and proximate to the distal end 707. The indentations 727 may be configured to have a predetermined depth, length, and/or curvature(s) to accommodate secure positioning and/or mating with one or more ball bearings of the adapter 704, as discussed herein. As can be understood, any other arrangement, depth, and/or number of the indentations 727 may be possible.

The adapter 704 may be configured as a quick connect mechanism and may include a quick connect collar 760, an inner sleeve housing 762 having a hollow housing 772 and the tool connector receiving opening 766, one or more latches 764 (which may be similar to latches 522, as shown in FIGS. 5a-c, in structure and/or function), and one or more openings 768 of a collar stopping mechanism. The hollow housing 772 and the tool connector receiving opening 766 may have diameters that may be greater than the diameter of the body 510 of the connector 702. This may allow the body 510 of the connector 502 to be inserted and securely coupled within the hollow housing 772. Moreover, the diameter of the hollow housing 772 may be larger than the outer diameter of the extension body 751 of the insert 750 to accommodate its insertion into the hollow housing 772 as shown in FIG. 7c. Secure coupling of the connector 502 within the hollow housing 772 may be accomplished using the latches 764 and the collar 760 that may be configured to prevent translational movements of the connector 502 once the latches 764 and the collar 760 are separately engaged. Translational movement of the connector 502 may be prevented upon the latches 764 engaging with the groove 714 of the connector 502. Rotational movement of the connector 502 may be prevented upon one or more ball bearings 780 engaging with one or more ball bearing indentations 727 of the insert 750.

To allow for rotational movement of the connector 702 and the insert 750 (if coupled thereto) (while preventing its translational movement), the collar 760 may be disengaged from the body 510 of the connector 502, for example, by the user pulling the collar 760 away from opening 766 (in a direction A, as shown in FIG. 7a). The movement of the collar 760 releases the ball bearings 780 from the indentations 727, thereby allowing the connector 502 and the insert 750 to rotate within the hollow interior 772 of the housing 762. To re-engage, the collar 760 may be released toward the opening 766, thereby engaging the ball bearings 780 with the indentations 727 of the insert 750. In some examples, the collar 760 may include a spring-loaded mechanism (e.g., a spring) (not shown in FIGS. 7a-b) that may be configured to push the collar 760 toward the opening 766 upon the user releasing it from being held away from the opening 766.

To allow for translational movement of the connector 502 (while preventing it rotational movement), the latches 764 (which may also be rotationally positioned about a pivot 792 within one or more corresponding cavities 776 and spring-loaded using a spring 795) may be pressed toward the interior of the housing 762 (e.g., in a direction B, as shown in FIG. 7a), thereby disengaging latches'teeth 797(a, b) from the groove 714. While the latches 764 are disengaged from the groove 714, the ball bearings 780 may still be engaged within the indentations 727 of the insert 750, which may allow movement of the connector 502 in and out of the hollow interior 772 (e.g., the connector 502 may be removed in its entirety from the hollow interior 772 and from the insert 750).

In some examples, to allow for both the translational and rotational movement of the connector 502 within the hollow interior 772, both latches 764 may be pressed toward the interior of the housing 762 (against the resistance of the spring 795) and the collar 760 may be pushed away from the opening 766. Such movements may fully release the connector 502 from the hollow interior 772 of the housing 762 as well as from the insert 750.

As shown in FIG. 7c (illustrating a cross-sectional view of the system 700 with the connector 502, insert 750, and the adapter 704 being coupled together), the adapter 704 may include a collar positioning portion 784 of the housing 762. The collar positioning portion 784 may include one or more ball bearing openings 786 that may be configured to accommodate positioning of one or more ball bearings 780. The openings 786 may be sized to allow the ball bearings 780 to extend toward the hollow interior 772 so that they can interact with one or more indentations 727 of the insert 750 while at the same time preventing the ball bearings 780 from falling through into the hollow interior 772. The collar 760 may be configured to secure the ball bearings 780 on an exterior surface of the portion 784 to prevent the ball bearings 780 from falling out in opposite direction (of the interior 772).

As discussed above, movement of the collar 760 along direction A (as shown in FIG. 7a), may be configured to engage (when the collar 760 is translated toward the opening 766) and disengage (when the collar 760 is translated away from the opening 766) the ball bearings 780 located within the indentations 727 of the insert 750, as shown in FIG. 7c.

The collar 760 may be moved between a locked or engaged position and an unlocked or disengaged position. In the engaged position, the collar 760, using its flat edge 796, may be configured to push one or more ball bearings 780 positioned inside their respective ball bearing openings 786 into the indentations 727 of the insert 750 positioned in the interior 772 and inserted over the body 710 of the connector 702. In the engaged position, the collar 760 along with the ball bearings 780 being engaged within the indentations 727 may be configured to prevent rotational motion of the connector 702.

To allow for rotational motion of the connector 502, the collar 760 may be pulled away from the opening 766. By pulling the collar 760, the flat edge 796 may be configured to be disengaged from the ball bearings 780, thereby allowing the ball bearings 780 to move inside their respective openings 786 and away from the indentations 727 of the insert 750 and thus, the connector 502.

As can be understood, there can be any number of ball bearings 780 and corresponding indentations 727 of the insert 750. In some exemplary, non-limiting implementations, the number of ball bearings 780 may be the same or different from the number of the indentations 727. Alternatively, or in addition, there can be fewer ball bearings 780 than the indentations 727.

Further, more or less ball bearings 780 may be used by increasing and/or decreasing diameters of the housing 762 and/or ball bearing sizes (e.g., more ball bearings may be fitted with a smaller ball bearing size and/or larger male/female diameters and vice versa). The ball bearing positioning in the housing 762 (and/or in the interior 772) may be configured to match the indentation 727 pattern of the insert 750 such that the desired rotational orientations may be configured to align and each ball bearing 780 may always have an indentation 727 of the insert 750 to seat into.

In some examples, use of the respective structures and geometries associated with one or more of the connector 502, the insert 750, and the adapter 704 may be configured to prevent translational and rotational movement of the connector 502, once the connector 502 is within the adapter 704 with the aid of the insert 750. Translational movement may be inhibited using interaction of the groove 514 of the connector 502 and the latches 764 of the adapter 704. Rotational movement may be prevented using the interaction between the insert's 750 protrusions 516 and the portion 508 of the connector 502. Moreover, once the insert 750 and the connector 502 are coupled together and inserted into the adapter 704, use of the ball bearings 780 and the indentations 727 of the insert 750 may prevent rotational movement of the connector 502 (and the insert 750) inside the adapter 704.

### IV. Impactor System

The following is a discussion of an exemplary, non-limiting orthopedic surgical instrument and/or impactor that may be used in connection with system 200, where such mechanisms may be used for quick-connecting of various tools, devices, instruments, implants, etc. As can be understood, the current subject matter is not limited to the use implementation of the impactor and/or any specific tools, and may be used in connection with any desired devices, mechanisms, tools, etc.

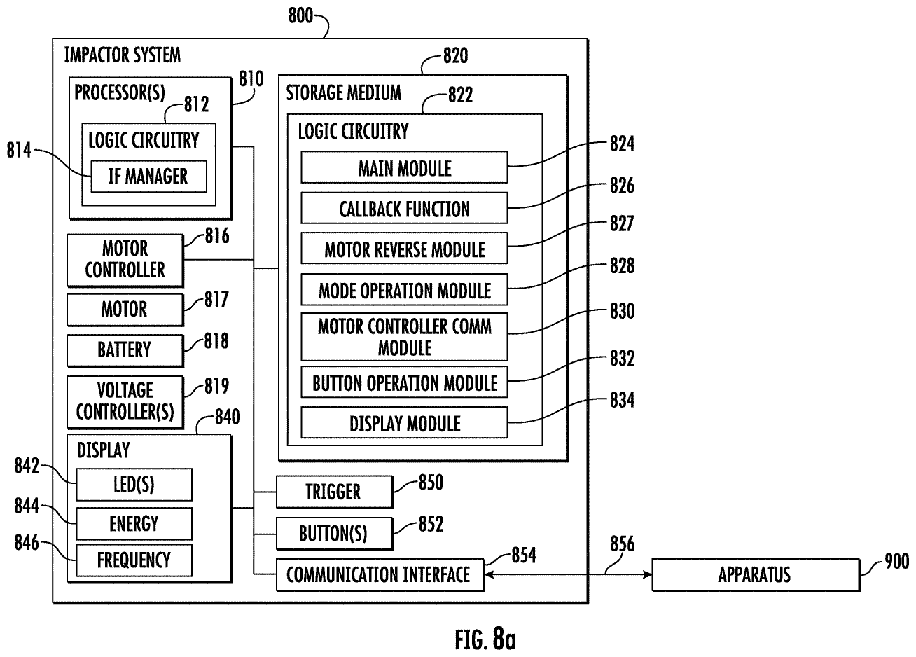

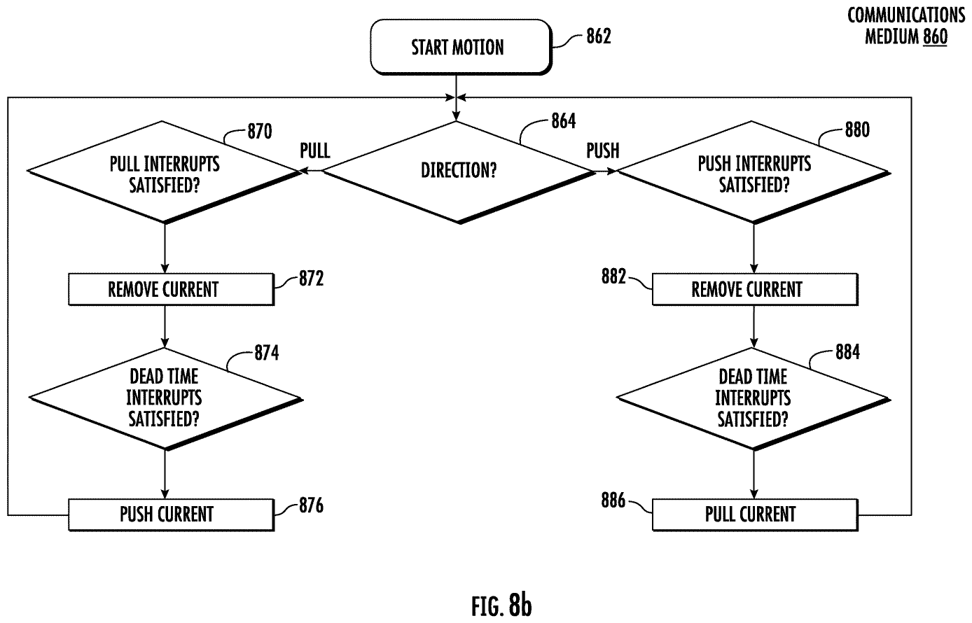

FIG. 8a is a block diagram of an exemplary orthopedic surgical instrument and/or impactor 800, according to some implementations of the current subject matter. FIG. 8b is a flowchart of an exemplary process 860 for operating of the orthopedic surgical instrument and/or impactor 800 shown in FIG. 8a, according to some implementations of the current subject matter. The impactor 800 may be similar to the impactor shown and described above in connection with FIGS. 1-7c.

The impactor 800 may combine with any suitable examples of the systems, devices, and methods disclosed herein. The impactor 800 may include processor(s) 810, a non-transitory storage medium 820, a motor controller 816, a motor 817, a battery 818, a voltage converter 819, a display 840, a trigger 850, button(s) 852, and a communication interface 854. The processor(s) 810 may include one or more processors, such as a programmable processor, a micro-controller unit (MCU), and/or the like. The processor(s) 810 may include processing circuitry to implement impactor logic circuitry 812 and 822.

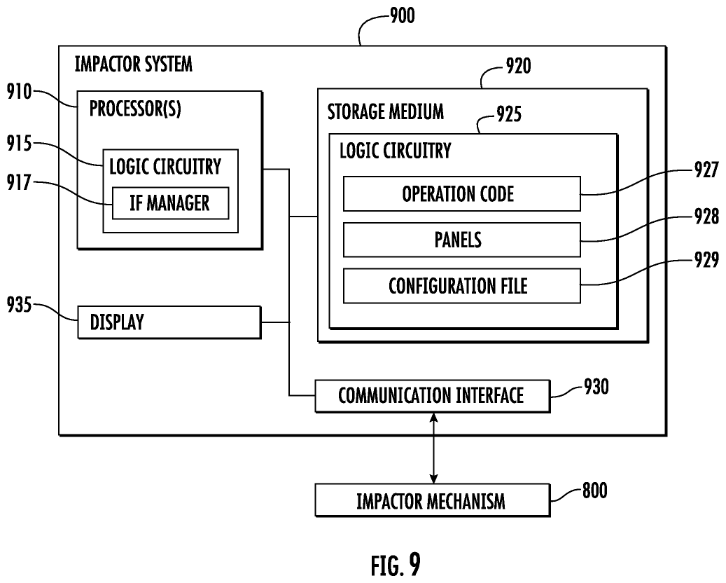

The processor(s) 810 may operatively couple with a non-transitory storage medium 820. The non-transitory storage medium 820 may store logic, code, and/or program instructions executable by the processor(s) 810 for performing one or more operations including the operations of the impactor logic circuitry 822. The non-transitory storage medium 820 may include one or more memory units (e.g., fixed or removable media or external storage such as a flash memory, secure digital (SD) card, random-access memory (RAM), read only memory (ROM), a flash drive, a hard drive, a solid-state drive (SSD) and/or the like). The memory units of the non-transitory storage medium 820 can store logic, code and/or program instructions executable by the processor(s) 810 to perform any suitable implementations of the current subject matter, as described herein. For example, the processor(s) 810 may execute instructions such as instructions of impactor logic circuitry 822 causing the motor 817 to operate the impactor at an impact energy and/or frequency selected by a user via button(s) 852 and/or via apparatus 900 (as shown in FIG. 9).

The processor(s) 810 may include code for the impactor 800 in memory within the processor(s) 810 and/or closely connected such as flash memory. The impactor logic circuitry 812 may represent code in or near the processor(s) 812 for execution by the processor(s) 810 and may include a user interface manager 814. The user interface manager 814 may include code executing on the processor(s) 810 to detect and respond to user input as well as to detect the motor controller 816 (such as, for example, a Maxon EPOS4 Controller) and establish communication with the motor controller 816.

The user interface manager 814 may communicate with the motor controller 816 to receive status information about the motor 817 and to control operation of the motor 817. For instance, all button presses of button(s) 852 and edit events may be posted to the user interface manager 814 and processed in real-time. The user interface manager 814 may communicate commands with the motor controller 816 to execute in response to the user's actions via button presses, system states, and error conditions. The user interface manager 814 may communicate alerts, warnings, and notifications to a user via the display 840 and or the apparatus 900 (as shown in FIG. 9) via the communications interface 854. Further, the user interface manager 814 may also handle user's response to alerts.

The motor 817 may include a DC motor, and/or any other motor. The battery 818 may include any desired power source.

The voltage converter(s) 819 may include a DC-DC voltage converters to adjust the voltage of signals to various voltages required to operate the components of the impactor 800 such as the processor(s) 810, the storage medium 820, and motor controller 816, the display 840, the trigger 850, the buttons 852, the communications interface 854, and/or the like.

The storage medium 820 may include a code for execution by the processor(s) 810 to operate the impactor 800. If desired, the processor(s) 810 may copy code from the storage medium 820 to memory closer to the processor(s) 810 to facilitate faster execution of the code. For instance, the user interface manager 814 may include code copied from the impactor logic circuitry 822 to memory closer to the processor(s) 810 for execution.

The impactor logic circuitry 822 may include code for operation of the impactor 800 stored in hardware of the storage medium such as volatile or non-volatile memory in the storage medium 820. The impactor logic circuitry 822 may include a main module 824, a callback module 826, a motor reverse module 827, a mode operation module 828, a motor controller communications module 830, a button operation module 832, and a display module 834.

The main module 824 may include setup and loop functions. The setup function may run once at start-up and the loop function may run continuously afterwards. The setup function may attach interrupts that run when button(s) 852 are pressed on the user interface, initializes Timer1 which runs the trigger interrupt service routine (ISR), and initializes an impact delay for the motor 817. The loop function allows the motor 817 to operate in the user-desired mode when the trigger 850 is enabled and pulled. The loop function also handles showing the user that the trigger state is enabled via LED(s) 842 of the display 840 and/or via the apparatus 900 (shown in FIG. 9).

The callback function 826 may be, e.g., an ISR that runs every millisecond. In some example implementations, the callback function 826 may run periodically with at a time period of more than one millisecond or less than one millisecond.

The motor reverse module 827 may include functions to prepare to reverse the motor 817, motor direction change of the motor 817, calculate impact delay of the impactor, and setup flutter time delays to set the frequency of impact while in flutter mode. These functions may switch the direction of the motor 817, reversing the motor 817 to allow for bi-directional operation of the impactor, and may also determine the delay between reversals for controlling a frequency of impacts of the impactor in a flutter mode.

The mode operation module 828 may include the functions of position check, flutter check, and oscillation check functions which are called for normal/full-swing mode, high-frequency/flutter mode, and oscillation mode respectively. Normal operation checks the position of the motor 817 then calls the prepare to reverse function. In some examples, the position of the motor 817 may be monitored via an encoder on a shaft of motor 817 that produces a count responsive to increments of rotation of the stator or shaft of the motor 817.

The motor controller communication module 828 may include the functions of enable motor controller 816 functions, set motor amperage (upper bound amperage), zero motor amperage (lower bound amperage), and disable the motor controller 816 functions. These functions communicate to the motor controller 816 whether or not to operate the motor 817 as well as set the operating amperage bounds for the motor 817.

The button operation module 832 may include functions to handle setting user-desired amperage and frequency to operate the motor 817 in addition to setting the operation mode and enabling the trigger 850. The functions may include energy plus to increase the energy of impact by the impactor, energy minus to increase the energy of impact by the impactor, frequency plus to increase the frequency of impacts by the impactor, frequency minus to decrease the frequency of impacts by the impactor, select operating mode to switch between available modes of operation (e.g., full-swing mode, flutter mode, or oscillation mode), and set trigger state to enable or disable the trigger 850. In some examples, these functions may be accessed via the apparatus 900 (shown in FIG. 9) and/or the button(s) 852. In alternate implementations, a touch screen may be included in the display in lieu of or in addition to the button(s) 852.

The display module 834 may include functions handle the logic for displaying the amperage and frequency on the user interface. The functions may include energy display and frequency display.

The display 840 may include LED(s) 840 and numerical, alphanumeric, or graphical displays such as LED displays or liquid crystal displays (LCDs) to present a number representative of the energy 844 and frequency 846 selected for operation of the motor 817. The button(s) 852 may include one or more buttons located in the display 840 and, In some examples, adjacent to the energy 844 and frequency 846 displays to provide a user with an interface to increase and/or decrease the energy and/or frequency of the impact of the impactor on the forward and/or the reverse motion.

The trigger 850 may include a trigger or other button or switch that, when actuated, can cause the impactor 800 to operate if the trigger 850 is enabled. If the trigger 850 is disabled, depressing the trigger 850 may not cause the impactor 800 to operate. In some examples, the trigger 850 cannot be depressed when the trigger 850 is disabled.

The processor(s) 810 may couple to a communication interface 854 to communicate with an apparatus 900 via a communications medium 856. The communications medium 856 may comprise a wired or wireless interface to communicatively coupled the impactor 800 with the apparatus 900 shown in FIG. 9.

The communication interface 854 may communicate user commands to and/or from the apparatus 900 to the impactor 800 to operate the impactor 800 via the functionality described in conjunction with the impactor 800. In some examples, the apparatus 800 may operate the motor 817 in addition to configuring parameters of operation of the motor 817 such as the upper current bound, the lower current bound, the operating current, the upper frequency bound, the lower frequency bound, the operating frequency, the mode of operation of the motor 817, and/or the like. In some examples, the communication interface 854 may communicate information about the operation of the impactor 800 to the apparatus 900 such as the energy of operation, the frequency of operation, the mode of operation, events or alerts associated with the impactor 800, and log information such as time and date of use, impact detections, encoder counts, and/or the like.

The communication interface 856 (and similarly, communication interface 930 shown in FIG. 9) may include circuitry to transmit and receive communications through a wired and/or wireless media such as an Ethernet interface, a wireless fidelity (Wi-Fi) interface, a cellular data interface, and/or the like. In some examples, the communication interface 856 (and/or interface 930) may implement logic such as code in a baseband processor to interact with a physical layer device to transmit and receive wireless communications from apparatus 900. For example, the communication interface 856 (and/or interface 930) may implement one or more of local area networks (LAN), wide area networks (WAN), infrared, radio, Wi-Fi, point-to-point (P2P) networks, telecommunication networks, cloud communication, and the like.

Referring to FIG. 8b, at 862, the process 860 may be initiated by starting motion of the motor. The motion may be started in any direction, at 864, either pulling or pushing the impactor.

In some examples, the impactor logic circuitry may monitor for a reduction in the count below a threshold or by a threshold deceleration of the counts. In some examples, the counts may vary based on a gear ration of the gear box coupled with the motor 817. The gear ratio may affect the granularity of the stator movement of the motor 817 per count, reducing the number of counts per stator rotation for gear boxes with low gear ratios such as 4.8:1 as compared with the number of counts per stator rotation for gear boxes with higher gear ratios such as 14:1. In such implementations, a threshold count may be different depending on the gear ratio of the gear box connected to the motor 817.

The impactor logic circuitry may determine if the number of interrupts received during pulling the hammer represent the selected number of interrupts, at 870. In some examples, the movement of the motor may be closely coupled with the movement of the impactor. The number of interrupts may represent the counts from the encoder of the motor or may represent counts of clock cycles so the impactor logic circuitry may determine whether the counts received at impact are within an expected range of counts for impact on the reverse. If the counts differ from the expected range of the counts, an event and/or alert may be generated to indicate a potential problem with the impactor 800.

If the number of interrupts are satisfied, the impactor logic circuitry may remove current from the motor 817, at 872 for a delay time (or dead time), at 874 that adjusts the frequency of impact of the hammer to a user selected frequency. The interrupts may represent clock cycles of a clock or may represent counts of stator movement determined to represent the amount of delay to include at the time of reversal of the motor 817 to set the frequency of impacts at the frequency selected by the user. In some examples, the interrupts may represent the callback function such as the callback function 826 shown in FIG. 8a and may, e.g., have a period of 1 millisecond. In other implementations, the period may be less than one millisecond or more than one millisecond.

Once the delay time is satisfied, the impactor logic circuitry may apply a push current to the motor 817, at 876, to rotate the stator of the motor and the shaft of the motor 817 in the opposite direction to push the impactor forward. For example, In some examples, a 9-ampere current may be a low energy setting and a 30-ampere current may be a high energy setting.

After applying the push current, the process 860 may return to 880 via 864. At 880, the impact logic circuitry may determine if the number of interrupts received at impact are within an expected range of counts for impact of the impactor on the forward impact. If the counts differ from the expected range of the counts, an event and/or alert may be generated to indicate a potential problem with the impactor 800.

If the number of push interrupts are satisfied, the impactor logic circuitry may remove current from the motor 817, at 882, for a delay time (or dead time), at 884 to adjust the frequency of impact of the hammer on the forward impact surface based on a user selected frequency. The interrupts may represent clock cycles of a clock or may represent counts of stator movement determined to represent the amount of delay time required at the time of reversal of the motor 817 to set the frequency of impacts at the frequency selected by the user. In some examples, the interrupts may represent the callback function such as the callback function 826 shown in FIG. 8a and may, e.g., have a period of 1 millisecond. In other implementations, the period may be less than one millisecond or more than one millisecond.

Once the delay time is satisfied, the impactor logic circuitry may apply a pull current to the motor 817, at 886, to rotate the stator of the motor and the shaft of the motor 817 in the opposite direction to pull the impactor backwards towards the reverse impact.

FIG. 9 illustrates an exemplary computing apparatus 900, according to some implementations of the current subject matter. The apparatus 900 may be a computing device that may be communicatively coupled with an orthopedic surgical instrument or impactor such as, orthopedic impactor 800 (e.g., as shown in FIG. 8a). The apparatus 900 may be a computer in the form of a smart phone, a tablet, a notebook, a desktop computer, a workstation, or a server. The apparatus 900 can combine with any suitable examples of the systems, devices, and methods disclosed herein. The apparatus 900 can include processor(s) 910, a non-transitory storage medium 920, communication interface 930, and a display 935. The processor(s) 910 may comprise one or more processors, such as a programmable processor (e.g., a central processing unit (CPU)). The processor(s) 910 may comprise processing circuitry to implement impactor logic circuitry 915 such as the impactor logic circuitry 812 shown in FIG. 8a.

The processor(s) 910 may include memory such as flash memory to contain program code for execution by the processor(s) 910. In some examples, the processor(s) 910 may have random access memory to contain a copy of code from flash memory or read only memory to facilitate faster execution of code. In some examples, the processor(s) 910 may include cache to contain data for faster calculations or execution. In some examples, the processor(s) 910 may include an impactor logic circuitry 915, which may include a user interface manager 917. The user interface manager 917 may function as a state machine controlled by keypad inputs, internal events or alarms, boundary conditions, exceptions and supervisory input to the user interface manager 917. The user interface manager 917 may process button presses and may update a main screen on the display 935 reflecting the state of the application.

Upon startup of the user interface manager 917, a handler may be installed to detect the motor controller 816 of the impactor 800 and to establish communication with the motor controller 816. In some examples, the button presses of button(s) 852 and edit events may be posted to a panel in the display 935 and may be processed in real-time. Motor controller commands may be executed upon the user's actions via button presses, system states, and error conditions. Further, the user interface manager 917 may implement alerts, warnings, and notifications and display the alerts, warnings, and notifications via the display 935. The user interface manager 917 may also include code to handle the user's response to alerts, warnings, and notifications.

The processor(s) 910 may operatively couple with a non-transitory storage medium 920. The non-transitory storage medium 920 may store logic, code, and/or program instructions executable by the processor(s) 910 for performing one or more instructions including the impactor logic circuitry 925. The non-transitory storage medium 920 may include one or more memory units (e.g., fixed and/or removable media or external storage such as electrically erasable programmable read only memory (EEPROM), a secure digital (SD) card, random-access memory (RAM), a flash drive, solid-state drive, a hard drive, and/or the like). The memory units of the non-transitory storage medium 920 may store logic, code and/or program instructions executable by the processor(s) 910 to perform any suitable implementation of the methods described herein. For example, the processor(s) 910 may execute instructions such as instructions of impactor logic circuitry 925 causing one or more processors of the processor(s) 910 to communicate user commands to the impactor 800 (as shown in FIG. 8a) and/or to communicate events, alerts, operation parameters for the impactor 800, and configurations.

The impactor logic circuitry 925 may include operation code 927, panels 928, and a configuration file 929. The operation code 927 may include functionality to set energy boundaries for operation of the impactor 800, set frequency boundaries for operation of the impactor 800, set an operating energy, set an operating frequency, set a impactor detection profile, set a boundary for a push current interrupt count, set a boundary for a pull current interrupt count, set a delay time or dead time interrupt count to establish a frequency of impact, set an operating mode (full swing, flutter, or oscillation), and/or the like.

The panels 928 may define graphical user interfaces for display of information and for receiving input parameters or configurations from a user. The configuration file 930 may include user selected parameters such as a motor controller with which to communicate, boundaries for energy (current), boundaries for frequency of impact, numbers of interrupts expected for push current and for pull current, and/or number of interrupts to receive to establish a frequency of impact.

The processor(s) 910 may couple to a communication interface 930 to transmit the data, code, or commands to and/or receive data, code, or commands from one or more external devices (e.g., a terminal, display device, a smart phone, a tablet, a server, or other remote device). The communication interface 930 includes circuitry to transmit and receive communications through a wired and/or wireless media such as an Ethernet interface, a wireless fidelity (Wi-Fi) interface, a Bluetooth interface such as a Bluetooth Low Energy (BLE) interface, a cellular data interface, and/or the like. In some examples, the communication interface 930 may implement logic such as code in a baseband processor to interact with a physical layer device to transmit and receive wireless communications from the impactor 800. For example, the communication interface 930 may implement one or more of local area networks (LAN), wide area networks (WAN), infrared, radio, Bluetooth, Wi-Fi, point-to-point (P2P) networks, telecommunication networks, cloud communication, and the like.

The processor(s) 910 may couple to a display 930 to display panels 928 for a user interface and/or other user interface items such as a message or notification via, graphics, video, text, and/or the like. In some examples, the display 930 may include a display on a terminal, a display device, a smart phone, a tablet, a server, or a remote device.

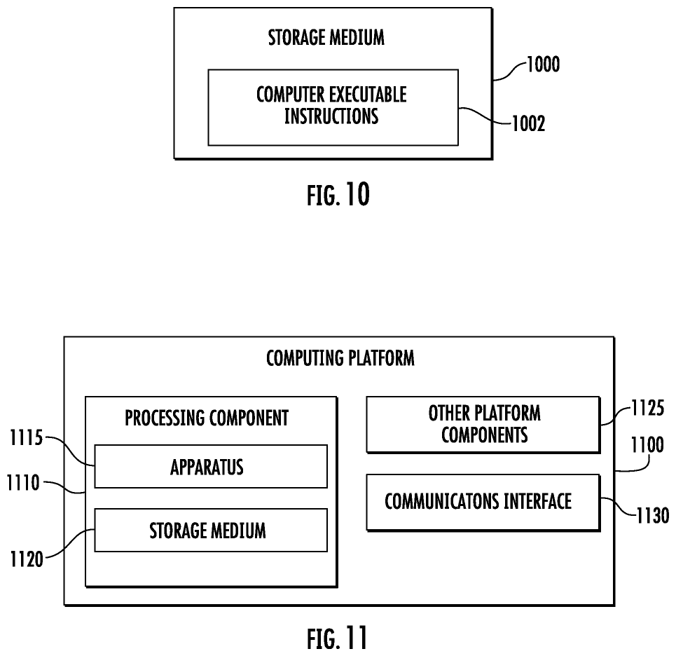

FIGS. 10-11 illustrate example implementations of a storage medium and computing platform for an orthopedic surgical instrument or impactor in accordance with one or more features of the present disclosure. FIG. 10 illustrates an example of a storage medium 1000 to store impactor logic. Storage medium 1000 may include an article of manufacture. In some examples, storage medium 1000 may include any non-transitory computer readable medium or machine readable medium, such as an optical, magnetic or semiconductor storage. Storage medium 1000 may store various types of computer executable instructions 1002, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 11 illustrates an example computing platform 1100. In some examples, as shown in FIG. 11, the computing platform 1100 may include a processing component 1110, other platform components or a communications interface 1130. According to some examples, computing platform 1100 may be implemented in a computing device such as a server in a system such as a data center or server farm that supports a manager or controller for managing configurable computing resources as mentioned above. Furthermore, the communications interface 1130 may include a wake-up radio (WUR) and may be capable of waking up a main radio of the computing platform 1100.

According to some examples, processing component 1110 may execute processing operations or logic for apparatus 1115 described herein such as the impactor logic circuitry 812, 915, and 925 illustrated in FIGS. 8a-b. Processing component 1110 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 1120, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 1125 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 1130 may include logic and/or features to support a communication interface. For these examples, communications interface 1130 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 1100 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 1100 described herein, may be included or omitted in various implementations of computing platform 1100, as suitably desired.

The components and features of computing platform 1100 may be implemented using any combination of discrete circuitry, ASICs, logic gates and/or single chip architectures. Further, the features of computing platform 1100 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the exemplary computing platform 1100 shown in the block diagram of FIG. 11 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in implementations.

One or more features of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

The foregoing description has broad application. While the present disclosure refers to certain implementations, numerous modifications, alterations, and changes to the described implementations are possible without departing from the sphere and scope of the present disclosure, as defined in the appended claim(s). Accordingly, it is intended that the present disclosure not be limited to the described implementations. Rather these implementations should be considered as illustrative and not restrictive in character. All changes and modifications that come within the spirit of the current subject matter are to be considered within the scope of the disclosure. The present disclosure should be given the full scope defined by the language of the following claims, and equivalents thereof. The discussion of any implementation is meant only to be explanatory and is not intended to suggest that the scope of the disclosure, including the claims, is limited to these implementations. In other words, while illustrative implementations of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counter-clockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular implementations. Such terms are not generally limiting to the scope of the claims made herein. Any implementation or feature of any section, portion, or any other component shown or particularly described in relation to various implementations of similar sections, portions, or components herein may be interchangeably applied to any other similar implementation or feature shown or described herein.

It should be understood that, as described herein, an “implementation” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated implementations are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one implementation” of the present disclosure are not intended to be interpreted as excluding the existence of additional implementations that also incorporate the recited features.

In addition, it will be appreciated that while the Figures may show one or more implementations of concepts or features together in a single implementation of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one implementation can be used separately, or with another implementation to yield a still further implementation. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “includes” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more implementations or configurations for the purpose of streamlining the disclosure. However, it should be understood that various features of the certain implementations or configurations of the disclosure may be combined in alternate implementations or configurations. Moreover, the following claims are hereby incorporated into this Detailed Description by this reference, with each claim standing on its own as a separate implementation of the present disclosure.

1. An apparatus for securing one or more external objects to a medical device, comprising:
a housing having a hollow interior;
a spring-loaded movable collar portion positioned exterior to the hollow interior of the housing; and
one or more spring-loaded latches coupled to the housing, the spring-loaded latches include one or more first retention mechanisms configured to releasably secure the one or more external objects within the hollow interior of the housing and prevent translational motion of the external objects once engaged by the spring-loaded latches;
one or more second retention mechanisms, wherein the spring-loaded movable collar portion includes one or more first portions of the one or more second retention mechanisms, and the housing includes one or more second portions of the one or more second retention mechanisms;
wherein upon interaction of the first and second portions of each of the one or more second retention mechanisms, the second retention mechanisms are configured to releasably secure the one or more external objects within the hollow interior of the housing and prevent rotational motion of the one or more external objects once engaged by the spring-loaded movable collar portion.

2. The apparatus of claim 1, wherein the one or more first portions of the one or more second retention mechanisms include one or more surfaces configured to interact with the one or more second portions of the second retention mechanism.

3. The apparatus of claim 1, wherein the one or more second portions of the one or more second retention mechanisms include one or more ball bearings secured within one or more openings in a wall of the housing.

4. The apparatus of claim 3, wherein the one or more ball bearings are configured to be temporarily positioned within the one or more openings in the wall of the housing and configured to releasably secure the one or more external objects.

5. The apparatus of claim 4, wherein the one or more external objects include one or more tool connectors.

6. The apparatus of claim 5, wherein the one or more tool connectors include a body having one or more grooves, wherein the one or more ball bearings are configured to be positioned within the one or more grooves upon the one or more tool connectors being positioned within the hollow interior of the housing and, thereby, prevent rotational motion of the one or more tool connectors.

7. The apparatus of claim 5, wherein the one or more tool connectors include one or more latch mating grooves.

8. The apparatus of claim 7, wherein the one or more spring-loaded latches include one or more connector engagement teeth, the one or more connector engagement teeth are configured to engage the one or more latch mating grooves upon the one or more tool connectors being positioned within the hollow interior of the housing, thereby, preventing translational motion of the one or more tool connectors being engaged by the spring-loaded latches.

9. The apparatus of claim 1, further comprising a spring configured to apply a pressure on the spring-loaded movable collar portion to retain the spring-loaded movable collar portion in a predetermined position to prevent the spring-loaded movable collar portion from releasing the one or more external objects from the interior portion of the housing.

10. The apparatus of claim 2, wherein the one or more spring-loaded latches are configured to be rotatably coupled within the interior portion of the housing.

11. The apparatus of claim 1, further comprising a latch spring configured to be coupled to the one or more spring-loaded latches.

12. The apparatus of claim 11, wherein the latch spring is configured to apply a pressure on the one or more spring-loaded latches to retain the one or more external objects within the interior portion of the housing.

13. The apparatus of claim 11, wherein the latch spring is configured to prevent the one or more spring-loaded latches from releasing the one or more external objects from within the interior portion of the housing.

14. The apparatus of claim 11, wherein the latch spring is configured to allow the one or more spring-loaded latches to permit a translational movement of the one or more external objects within the interior portion of the housing.

15. The apparatus of claim 1, wherein the medical device is an orthopedic surgical impactor.

16. The apparatus of claim 1, wherein the one or more external objects include at least one of the following: a tool, an instrument, a device, a drill, a cutting tool, an effector, a broach, an implant, and any combination thereof.

17-32. (canceled)

33. An apparatus for securing one or more external objects to a medical device, comprising:
one or more connectors;
an adapter mechanism, the adapter mechanism including:
a housing having a hollow interior,
one or more spring-loaded latches coupled to the housing, the one or more spring-loaded latches having one or more first retention mechanisms configured to releasably secure the one or more connectors within the hollow interior of the housing and prevent translational motion of the one or more connectors once engaged by the one or more spring-loaded latches; and
one or more second retention mechanisms, wherein the one or more connectors are configured to include one or more adapter locking portions configured to interact with the one or more second retention mechanisms to prevent rotational motion of the one or more connectors once the one or more connectors are engaged by the one or more second retention mechanisms.

34. The apparatus of claim 33, wherein the one or more second retention mechanisms include one or more protrusions and one or more grooves positioned between the one or more protrusions;
wherein, upon an interaction of the one or more adapter locking portions of the one or more connectors and the one or more second retention mechanisms, the one or more grooves are configured to receive the one or more adapter locking portions such that the one or more adapter locking portions are positioned between the one or more protrusions.

35. The apparatus of claim 33, further comprising one or more inserts positioned between the adapter mechanism and the one or more connectors.

36. The apparatus of claim 35, wherein the one or more inserts are configured to be at least partially positioned within an interior portion of the adapter mechanism.

37. The apparatus of claim 35, wherein the one or more first retention mechanisms include one or more ball bearings secured within one or more openings in a wall of the housing.

38. The apparatus of claim 37, wherein the one or more ball bearings are configured to be temporarily positioned within the one or more openings in the wall of the housing for releasably securing of the one or more connectors.

39. The apparatus of claim 35, wherein the one or more ball bearings are configured to be positioned within one or more indentations of the one or more inserts and configured to prevent rotational motion of the one or more connectors once the one or more connectors are engaged by the one or more indentations of the one or more inserts.

40. The apparatus of claim 32, wherein the one or more spring-loaded latches are rotatably coupled within the interior portion of the housing.
